SEQUENCE LISTING (207 sequences)

SEQ ID NO: 1 - DNA, 23 bp, synthetic construct
  misc_feature 1..23: note = Forward primer sequence for rDNA 16S hypervariable region 1
  source 1..23: mol_type = other DNA
tttagagtttgatcmtggctcag

SEQ ID NO: 2 - DNA, 22 bp, synthetic construct
  misc_feature 1..22: note = Reverse primer sequence for rDNA 16S hypervariable region 1
  source 1..22: mol_type = other DNA
tttacattactcacccgtccgc

SEQ ID NO: 3 - DNA, 22 bp, synthetic construct
  misc_feature 1..22: note = Reverse primer sequence for rDNA 16S hypervariable region 1
  source 1..22: mol_type = other DNA
tttgtgttactcacccgtycgc

SEQ ID NO: 4 - DNA, 24 bp, synthetic construct
  misc_feature 1..24: note = Forward primer sequence for rDNA 16S hypervariable region 3
  source 1..24: mol_type = other DNA
tttcacattggractgagacacgg

SEQ ID NO: 5 - DNA, 25 bp, synthetic construct
  misc_feature 1..25: note = Reverse primer sequence for rDNA 16S hypervariable region 3
  source 1..25: mol_type = other DNA
ttttgtattaccgcggctgctggca

SEQ ID NO: 6 - DNA, 23 bp, synthetic construct
  misc_feature 1..23: note = Forward primer sequence for rDNA 16S hypervariable region 6
  source 1..23: mol_type = other DNA
ttcgcacaagcggtggagcatgt

SEQ ID NO: 7 - DNA, 25 bp, synthetic construct
  misc_feature 1..25: note = Reverse primer sequence for rDNA 16S hypervariable region 6
  source 1..25: mol_type = other DNA
tttacgagctgacgacagccatgca

SEQ ID NO: 8 - DNA, 22 bp, synthetic construct
  misc_feature 1..22: note = Forward primer sequence for rDNA 28S D2 region
  source 1..22: mol_type = other DNA
ttgtacgtgaaattgttgaaag

SEQ ID NO: 9 - DNA, 20 bp, synthetic construct
  misc_feature 1..20: note = Reverse primer sequence for rDNA 28S D2 region
  source 1..20: mol_type = other DNA
ttaaaccgcagtcctcrgtc

SEQ ID NO: 10 - DNA, 28 bp, synthetic construct
  misc_feature 1..28: note = Forward primer sequence for human RNaseP
  source 1..28: mol_type = other DNA
tttgtttgcagatttggacctgcgagcg

SEQ ID NO: 11 - DNA, 28 bp, synthetic construct
  misc_feature 1..28: note = Reverse primer sequence for for human RNaseP
  source 1..28: mol_type = other DNA
tttaaggtgagcggctgtctccacaagt

SEQ ID NO: 12 - DNA, 30 bp, synthetic construct
  misc_feature 1..30: note = Probe sequence for bacterial rDNA 16s hypervariable region 1a
  source 1..30: mol_type = other DNA
tttttttgaaccgacgaagtgctctttttt

SEQ ID NO: 13 - DNA, 30 bp, synthetic construct
  misc_feature 1..30: note = Probe sequence for bacterial rDNA 16s hypervariable region 1a
  source 1..30: mol_type = other DNA
tttttattgggaaggtagcttgtatttctt

SEQ ID NO: 14 - DNA, 30 bp, synthetic construct
  misc_feature 1..30: note = Probe sequence for bacterial rDNA 16s hypervariable region 1a
  source 1..30: mol_type = other DNA
tttttttatgcacagaggagctatttattt

SEQ ID NO: 15 - DNA, 30 bp, synthetic construct
  misc_feature 1..30: note = Probe sequence for bacterial rDNA 16s hypervariable region 1a
  source 1..30: mol_type = other DNA
tttttctacaggaagcagcttgcctttttt

SEQ ID NO: 16 - DNA, 30 bp, synthetic construct
  misc_feature 1..30: note = Probe sequence for bacterial rDNA 16s hypervariable region 1a
  source 1..30: mol_type = other DNA
tttttttcagcacagagagcttattttttt

SEQ ID NO: 17 - DNA, 30 bp, synthetic construct
  misc_feature 1..30: note = Probe sequence for bacterial rDNA 16s hypervariable region 1a
  source 1..30: mol_type = other DNA
tttttgcttctttcctcccgagtgtctttt

SEQ ID NO: 18 - DNA, 30 bp, synthetic construct
  misc_feature 1..30: note = Probe sequence for bacterial rDNA 16s hypervariable region 1a
  source 1..30: mol_type = other DNA
ttttcgcttctttttccaccggagtctttt

SEQ ID NO: 19 - DNA, 30 bp, synthetic construct
  misc_feature 1..30: note = Probe sequence for bacterial rDNA 16s hypervariable region 1a
  source 1..30: mol_type = other DNA
tttttattaacagggagaagctattttttt

SEQ ID NO: 20 - DNA, 30 bp, synthetic construct
  misc_feature 1..30: note = Probe sequence for bacterial rDNA 16s hypervariable region 1a
  source 1..30: mol_type = other DNA
tttttagtaacagragaaagctctattttt

SEQ ID NO: 21 - DNA, 30 bp, synthetic construct
  misc_feature 1..30: note = Probe sequence for bacterial rDNA 16s hypervariable region 1a
  source 1..30: mol_type = other DNA
tttatttggtaacagaagaaagcatttttt

SEQ ID NO: 22 - DNA, 30 bp, synthetic construct
  misc_feature 1..30: note = Probe sequence for bacterial rDNA 16s hypervariable region 1a
  source 1..30: mol_type = other DNA
tttttttttaacaggggaagcttattattt

SEQ ID NO: 23 - DNA, 30 bp, synthetic construct
  misc_feature 1..30: note = Probe sequence for bacterial rDNA 16s hypervariable region 1a
  source 1..30: mol_type = other DNA
tttttactggatgaagggagctattttttt

SEQ ID NO: 24 - DNA, 30 bp, synthetic construct
  misc_feature 1..30: note = Probe sequence for bacterial rDNA 16s hypervariable region 1a
  source 1..30: mol_type = other DNA
tttttatcttatgaagggagctattttttt

SEQ ID NO: 25 - DNA, 30 bp, synthetic construct
  misc_feature 1..30: note = Probe sequence for bacterial rDNA 16s hypervariable region 1a
  source 1..30: mol_type = other DNA
ttttttttacggacgagaagcccttttttt

SEQ ID NO: 26 - DNA, 30 bp, synthetic construct
  misc_feature 1..30: note = Probe sequence for bacterial rDNA 16s hypervariable region 1a
  source 1..30: mol_type = other DNA
tttttttgaacagataaggagctatttttt

SEQ ID NO: 27 - DNA, 30 bp, synthetic construct
  misc_feature 1..30: note = Probe sequence for bacterial rDNA 16s hypervariable region 1a
  source 1..30: mol_type = other DNA
tttttttggtttggtgtttacatctttttt

SEQ ID NO: 28 - DNA, 30 bp, synthetic construct
  misc_feature 1..30: note = Probe sequence for bacterial rDNA 16s hypervariable region 1a
  source 1..30: mol_type = other DNA
tttttttctgcacaggggagctattttttt

SEQ ID NO: 29 - DNA, 30 bp, synthetic construct
  misc_feature 1..30: note = Probe sequence for bacterial rDNA 16s hypervariable region 1a
  source 1..30: mol_type = other DNA
ttttttttatcggaagtagcaatttttttt

SEQ ID NO: 30 - DNA, 30 bp, synthetic construct
  misc_feature 1..30: note = Probe sequence for bacterial rDNA 16s hypervariable region 1a
  source 1..30: mol_type = other DNA
tttttttgagcgaggttagcaatttttttt

SEQ ID NO: 31 - DNA, 30 bp, synthetic construct
  misc_feature 1..30: note = Probe sequence for bacterial rDNA 16s hypervariable region 1a
  source 1..30: mol_type = other DNA
tttttctcgaacgaagccttttagtttttt

SEQ ID NO: 32 - DNA, 30 bp, synthetic construct
  misc_feature 1..30: note = Probe sequence for bacterial rDNA 16s hypervariable region 1c
  source 1..30: mol_type = other DNA
tttttttttgcacttctgacgtattttttt

SEQ ID NO: 33 - DNA, 30 bp, synthetic construct
  misc_feature 1..30: note = Probe sequence for bacterial rDNA 16s hypervariable region 1c
  source 1..30: mol_type = other DNA
tttttttctgctaccggacctatttttttt

SEQ ID NO: 34 - DNA, 30 bp, synthetic construct
  misc_feature 1..30: note = Probe sequence for bacterial rDNA 16s hypervariable region 1c
  source 1..30: mol_type = other DNA
ttttttttcttgctccttgggttttttttt

SEQ ID NO: 35 - DNA, 30 bp, synthetic construct
  misc_feature 1..30: note = Probe sequence for bacterial rDNA 16s hypervariable region 1c
  source 1..30: mol_type = other DNA
tttttttcttgctgctttgctgattttttt

SEQ ID NO: 36 - DNA, 30 bp, synthetic construct
  misc_feature 1..30: note = Probe sequence for bacterial rDNA 16s hypervariable region 1c
  source 1..30: mol_type = other DNA
ttttttttcttgctctcgggtttttttttt

SEQ ID NO: 37 - DNA, 30 bp, synthetic construct
  misc_feature 1..30: note = Probe sequence for bacterial rDNA 16s hypervariable region 1c
  source 1..30: mol_type = other DNA
ttttgcttgcactcaattggaaagtctttt

SEQ ID NO: 38 - DNA, 30 bp, synthetic construct
  misc_feature 1..30: note = Probe sequence for bacterial rDNA 16s hypervariable region 1c
  source 1..30: mol_type = other DNA
ttctttgcttgctccaccggaaaacttttt

SEQ ID NO: 39 - DNA, 30 bp, synthetic construct
  misc_feature 1..30: note = Probe sequence for bacterial rDNA 16s hypervariable region 1c
  source 1..30: mol_type = other DNA
ttttttctcttctctgctgacgtttttttt

SEQ ID NO: 40 - DNA, 30 bp, synthetic construct
  misc_feature 1..30: note = Probe sequence for bacterial rDNA 16s hypervariable region 1c
  source 1..30: mol_type = other DNA
tttttttctgctttcttgctgacttttttt

SEQ ID NO: 41 - DNA, 30 bp, synthetic construct
  misc_feature 1..30: note = Probe sequence for bacterial rDNA 16s hypervariable region 1c
  source 1..30: mol_type = other DNA
ttttttttttgcttctcgctgatttttttt

SEQ ID NO: 42 - DNA, 30 bp, synthetic construct
  misc_feature 1..30: note = Probe sequence for bacterial rDNA 16s hypervariable region 1c
  source 1..30: mol_type = other DNA
tttttttttgctcctggattcatttttttt

SEQ ID NO: 43 - DNA, 30 bp, synthetic construct
  misc_feature 1..30: note = Probe sequence for bacterial rDNA 16s hypervariable region 1c
  source 1..30: mol_type = other DNA
ttttttttcttgccttggattcattttttt

SEQ ID NO: 44 - DNA, 30 bp, synthetic construct
  misc_feature 1..30: note = Probe sequence for bacterial rDNA 16s hypervariable region 1c
  source 1..30: mol_type = other DNA
ttttttctgcttctctgatgttattttttt

SEQ ID NO: 45 - DNA, 30 bp, synthetic construct
  misc_feature 1..30: note = Probe sequence for bacterial rDNA 16s hypervariable region 1c
  source 1..30: mol_type = other DNA
tttttttctgctcctttgacgttttttttt

SEQ ID NO: 46 - DNA, 30 bp, synthetic construct
  misc_feature 1..30: note = Probe sequence for bacterial rDNA 16s hypervariable region 1c
  source 1..30: mol_type = other DNA
ttttttcgtttacactagactgtccttttt

SEQ ID NO: 47 - DNA, 30 bp, synthetic construct
  misc_feature 1..30: note = Probe sequence for bacterial rDNA 16s hypervariable region 1c
  source 1..30: mol_type = other DNA
ttttttttcttgctccctgggttttttttt

SEQ ID NO: 48 - DNA, 30 bp, synthetic construct
  misc_feature 1..30: note = Probe sequence for bacterial rDNA 16s hypervariable region 1c
  source 1..30: mol_type = other DNA
tttttttgtagcaatactttagattttttt

SEQ ID NO: 49 - DNA, 30 bp, synthetic construct
  misc_feature 1..30: note = Probe sequence for bacterial rDNA 16s hypervariable region 1c
  source 1..30: mol_type = other DNA
ttttttccagcaataacctagcggtttttt

SEQ ID NO: 50 - DNA, 30 bp, synthetic construct
  misc_feature 1..30: note = Probe sequence for bacterial rDNA 16s hypervariable region 1c
  source 1..30: mol_type = other DNA
tttttttgcttagtggtgaacgcttttttt

SEQ ID NO: 51 - DNA, 30 bp, synthetic construct
  misc_feature 1..30: note = Probe sequence for bacterial rDNA 16s hypervariable region 3a
  source 1..30: mol_type = other DNA
tttttttacaaattggagagtaatcttttt

SEQ ID NO: 52 - DNA, 32 bp, synthetic construct
  misc_feature 1..32: note = Probe sequence for bacterial rDNA 16s hypervariable region 3a
  source 1..32: mol_type = other DNA
tttttttgctactttagttaatactctttttt

SEQ ID NO: 53 - DNA, 30 bp, synthetic construct
  misc_feature 1..30: note = Probe sequence for bacterial rDNA 16s hypervariable region 3a
  source 1..30: mol_type = other DNA
ttttttacaggaaggcgttgtgtatttttt

SEQ ID NO: 54 - DNA, 30 bp, synthetic construct
  misc_feature 1..30: note = Probe sequence for bacterial rDNA 16s hypervariable region 3a
  source 1..30: mol_type = other DNA
ttttttcagggagtaaagttaatatttttt

SEQ ID NO: 55 - DNA, 30 bp, synthetic construct
  misc_feature 1..30: note = Probe sequence for bacterial rDNA 16s hypervariable region 3a
  source 1..30: mol_type = other DNA
tttttttgaaggcgttaaggttatcttttt

SEQ ID NO: 56 - DNA, 30 bp, synthetic construct
  misc_feature 1..30: note = Probe sequence for bacterial rDNA 16s hypervariable region 3a
  source 1..30: mol_type = other DNA
ttttttgaacaaggacgttagtaatttttt

SEQ ID NO: 57 - DNA, 30 bp, synthetic construct
  misc_feature 1..30: note = Probe sequence for bacterial rDNA 16s hypervariable region 3a
  source 1..30: mol_type = other DNA
tttttttcaaggatgagagtaacctttttt

SEQ ID NO: 58 - DNA, 32 bp, synthetic construct
  misc_feature 1..32: note = Probe sequence for bacterial rDNA 16s hypervariable region 3a
  source 1..32: mol_type = other DNA
ttttttttaaggtgtcaaggttaaattttttt

SEQ ID NO: 59 - DNA, 30 bp, synthetic construct
  misc_feature 1..30: note = Probe sequence for bacterial rDNA 16s hypervariable region 3a
  source 1..30: mol_type = other DNA
ttttttgtgataaggttaatacccattttt

SEQ ID NO: 60 - DNA, 30 bp, synthetic construct
  misc_feature 1..30: note = Probe sequence for bacterial rDNA 16s hypervariable region 3a
  source 1..30: mol_type = other DNA
tttttgtgataaagttaataccttcttttt

SEQ ID NO: 61 - DNA, 30 bp, synthetic construct
  misc_feature 1..30: note = Probe sequence for bacterial rDNA 16s hypervariable region 3a
  source 1..30: mol_type = other DNA
tttttttaaggcgttgatgttaaatttttt

SEQ ID NO: 62 - DNA, 32 bp, synthetic construct
  misc_feature 1..32: note = Probe sequence for bacterial rDNA 16s hypervariable region 3a
  source 1..32: mol_type = other DNA
tttttttcaagggcagtaagttaaattttttt

SEQ ID NO: 63 - DNA, 30 bp, synthetic construct
  misc_feature 1..30: note = Probe sequence for bacterial rDNA 16s hypervariable region 3a
  source 1..30: mol_type = other DNA
ttttttgaagaacatatgtgtaagcttttt

SEQ ID NO: 64 - DNA, 30 bp, synthetic construct
  misc_feature 1..30: note = Probe sequence for bacterial rDNA 16s hypervariable region 3a
  source 1..30: mol_type = other DNA
ttttctaagaacaaaygtgtaagctttttt

SEQ ID NO: 65 - DNA, 32 bp, synthetic construct
  misc_feature 1..32: note = Probe sequence for bacterial rDNA 16s hypervariable region 3a
  source 1..32: mol_type = other DNA
ttttttttagaacgttggtaggattttttttt

SEQ ID NO: 66 - DNA, 30 bp, synthetic construct
  misc_feature 1..30: note = Probe sequence for bacterial rDNA 16s hypervariable region 3a
  source 1..30: mol_type = other DNA
tttttttaggtggtgarcttaatttttttt

SEQ ID NO: 67 - DNA, 30 bp, synthetic construct
  misc_feature 1..30: note = Probe sequence for bacterial rDNA 16s hypervariable region 3a
  source 1..30: mol_type = other DNA
tttttcaagaatgactctagcagttttttt

SEQ ID NO: 68 - DNA, 32 bp, synthetic construct
  misc_feature 1..32: note = Probe sequence for bacterial rDNA 16s hypervariable region 3a
  source 1..32: mol_type = other DNA
tttttttcagaaacgctaagatagttcttttt

SEQ ID NO: 69 - DNA, 32 bp, synthetic construct; identical to SEQ ID NO: 68
  misc_feature 1..32: note = Probe sequence for bacterial rDNA 16s hypervariable region 3a
  source 1..32: mol_type = other DNA

SEQ ID NO: 70 - DNA, 30 bp, synthetic construct
  misc_feature 1..30: note = Probe sequence for bacterial rDNA 16s hypervariable region 3a
  source 1..30: mol_type = other DNA
tttttcttggaaggtgttgtgtatttcttt

SEQ ID NO: 71 - DNA, 32 bp, synthetic construct
  misc_feature 1..32: note = Probe sequence for bacterial rDNA 16s hypervariable region 3a
  source 1..32: mol_type = other DNA
ttttttctaagggagtgaggttaaatcttttt

SEQ ID NO: 72 - DNA, 30 bp, synthetic construct
  misc_feature 1..30: note = Probe sequence for bacterial rDNA 16s hypervariable region 3a
  source 1..30: mol_type = other DNA
ttttttttgaaggcgatgaggttttttttt

SEQ ID NO: 73 - DNA, 30 bp, synthetic construct
  misc_feature 1..30: note = Probe sequence for bacterial rDNA 16s hypervariable region 3a
  source 1..30: mol_type = other DNA
tttttttaaggcgataaggttaattttttt

SEQ ID NO: 74 - DNA, 30 bp, synthetic construct
  misc_feature 1..30: note = Probe sequence for bacterial rDNA 16s hypervariable region 3a
  source 1..30: mol_type = other DNA
ttttttctaaggcggtgaggtttttttttt

SEQ ID NO: 75 - DNA, 30 bp, synthetic construct
  misc_feature 1..30: note = Probe sequence for bacterial rDNA 16s hypervariable region 3a
  source 1..30: mol_type = other DNA
tttttctatgtgagttaagttacttttttt

SEQ ID NO: 76 - DNA, 30 bp, synthetic construct
  misc_feature 1..30: note = Probe sequence for bacterial rDNA 16s hypervariable region 3c
  source 1..30: mol_type = other DNA
tttttttcaactgctccagtcttttttttt

SEQ ID NO: 77 - DNA, 32 bp, synthetic construct
  misc_feature 1..32: note = Probe sequence for bacterial rDNA 16s hypervariable region 3c
  source 1..32: mol_type = other DNA
ttttttctatacctagagatagtgtttttttt

SEQ ID NO: 78 - DNA, 30 bp, synthetic construct
  misc_feature 1..30: note = Probe sequence for bacterial rDNA 16s hypervariable region 3c
  source 1..30: mol_type = other DNA
ttttttttgcagcgattgacgtattttttt

SEQ ID NO: 79 - DNA, 32 bp, synthetic construct
  misc_feature 1..32: note = Probe sequence for bacterial rDNA 16s hypervariable region 3c
  source 1..32: mol_type = other DNA
tttttttttacctttgctcattgatttttttt

SEQ ID NO: 80 - DNA, 32 bp, synthetic construct
  misc_feature 1..32: note = Probe sequence for bacterial rDNA 16s hypervariable region 3c
  source 1..32: mol_type = other DNA
tttttttttaaccttggcgattgaattttttt

SEQ ID NO: 81 - DNA, 30 bp, synthetic construct
  misc_feature 1..30: note = Probe sequence for bacterial rDNA 16s hypervariable region 3c
  source 1..30: mol_type = other DNA
tttttttaactgaacgtcccctgatttttt

SEQ ID NO: 82 - DNA, 30 bp, synthetic construct
  misc_feature 1..30: note = Probe sequence for bacterial rDNA 16s hypervariable region 3c
  source 1..30: mol_type = other DNA
ttttttttaactgttcatcccttgtttttt

SEQ ID NO: 83 - DNA, 30 bp, synthetic construct
  misc_feature 1..30: note = Probe sequence for bacterial rDNA 16s hypervariable region 3c
  source 1..30: mol_type = other DNA
ttttttttaaccttggcaattgactttttt

SEQ ID NO: 84 - DNA, 30 bp, synthetic construct
  misc_feature 1..30: note = Probe sequence for bacterial rDNA 16s hypervariable region 3c
  source 1..30: mol_type = other DNA
ttttttggttaatacccttrtcatcttttt

SEQ ID NO: 85 - DNA, 30 bp, synthetic construct
  misc_feature 1..30: note = Probe sequence for bacterial rDNA 16s hypervariable region 3c
  source 1..30: mol_type = other DNA
ttttctagttaatacctttgtcatcttttt

SEQ ID NO: 86 - DNA, 30 bp, synthetic construct
  misc_feature 1..30: note = Probe sequence for bacterial rDNA 16s hypervariable region 3c
  source 1..30: mol_type = other DNA
ttttttttaccatcaacgattgattttttt

SEQ ID NO: 87 - DNA, 30 bp, synthetic construct
  misc_feature 1..30: note = Probe sequence for bacterial rDNA 16s hypervariable region 3c
  source 1..30: mol_type = other DNA
tttttctataccttgctgttttgatttttt

SEQ ID NO: 88 - DNA, 32 bp, synthetic construct
  misc_feature 1..32: note = Probe sequence for bacterial rDNA 16s hypervariable region 3c
  source 1..32: mol_type = other DNA
ttttttctataccttgctgtttgatttttttt

SEQ ID NO: 89 - DNA, 30 bp, synthetic construct
  misc_feature 1..30: note = Probe sequence for bacterial rDNA 16s hypervariable region 3c
  source 1..30: mol_type = other DNA
ttttttttgtgcacatcttgactttttttt

SEQ ID NO: 90 - DNA, 30 bp, synthetic construct
  misc_feature 1..30: note = Probe sequence for bacterial rDNA 16s hypervariable region 3c
  source 1..30: mol_type = other DNA
tttttttaactgtgtacgtcttgttttttt

SEQ ID NO: 91 - DNA, 30 bp, synthetic construct
  misc_feature 1..30: note = Probe sequence for bacterial rDNA 16s hypervariable region 3c
  source 1..30: mol_type = other DNA
tttcttcggaaaatctaccaagtgtttttt

SEQ ID NO: 92 - DNA, 30 bp, synthetic construct
  misc_feature 1..30: note = Probe sequence for bacterial rDNA 16s hypervariable region 3c
  source 1..30: mol_type = other DNA
tttttcaatacgytcatcaattgaattttt

SEQ ID NO: 93 - DNA, 30 bp, synthetic construct
  misc_feature 1..30: note = Probe sequence for bacterial rDNA 16s hypervariable region 3c
  source 1..30: mol_type = other DNA
ttttttaggctggagtttgacttttttttt

SEQ ID NO: 94 - DNA, 32 bp, synthetic construct
  misc_feature 1..32: note = Probe sequence for bacterial rDNA 16s hypervariable region 3c
  source 1..32: mol_type = other DNA
tttttttcgattgcagactgacgttttttttt

SEQ ID NO: 95 - DNA, 32 bp, synthetic construct
  misc_feature 1..32: note = Probe sequence for bacterial rDNA 16s hypervariable region 3c
  source 1..32: mol_type = other DNA
tttttttaatgattttagtttgactttttttt

SEQ ID NO: 96 - DNA, 30 bp, synthetic construct
  misc_feature 1..30: note = Probe sequence for bacterial rDNA 16s hypervariable region 3c
  source 1..30: mol_type = other DNA
ttttttttcgcagcaattgacgcttttttt

SEQ ID NO: 97 - DNA, 32 bp, synthetic construct
  misc_feature 1..32: note = Probe sequence for bacterial rDNA 16s hypervariable region 3c
  source 1..32: mol_type = other DNA
ttttttttaaccttattcattgactctttttt

SEQ ID NO: 98 - DNA, 30 bp, synthetic construct
  misc_feature 1..30: note = Probe sequence for bacterial rDNA 16s hypervariable region 3c
  source 1..30: mol_type = other DNA
tttttctataacctcatcgattgttttttt

SEQ ID NO: 99 - DNA, 30 bp, synthetic construct
  misc_feature 1..30: note = Probe sequence for bacterial rDNA 16s hypervariable region 3c
  source 1..30: mol_type = other DNA
ttttttttaaccttgtcgattgaatttttt

SEQ ID NO: 100 - DNA, 30 bp, synthetic construct
  misc_feature 1..30: note = Probe sequence for bacterial rDNA 16s hypervariable region 3c
  source 1..30: mol_type = other DNA
ttttttcactaccttagctcatattttttt

SEQ ID NO: 101 - DNA, 30 bp, synthetic construct
  misc_feature 1..30: note = Probe sequence for bacterial rDNA 16s hypervariable region 3e
  source 1..30: mol_type = other DNA
ttctttaggtatcttaccagaaactttttt

SEQ ID NO: 102 - DNA, 30 bp, synthetic construct
  misc_feature 1..30: note = Probe sequence for bacterial rDNA 16s hypervariable region 3e
  source 1..30: mol_type = other DNA
ttttttctacgttactcgcagaattttttt

SEQ ID NO: 103 - DNA, 30 bp, synthetic construct
  misc_feature 1..30: note = Probe sequence for bacterial rDNA 16s hypervariable region 3e
  source 1..30: mol_type = other DNA
ttttttttcggtatctaaccagactttttt

SEQ ID NO: 104 - DNA, 30 bp, synthetic construct
  misc_feature 1..30: note = Probe sequence for bacterial rDNA 16s hypervariable region 3e
  source 1..30: mol_type = other DNA
ttttttctacgttacccgcagaattttttt

SEQ ID NO: 105 - DNA, 30 bp, synthetic construct
  misc_feature 1..30: note = Probe sequence for bacterial rDNA 16s hypervariable region 3e
  source 1..30: mol_type = other DNA
ttttttttgttaccgacagaagattttttt

SEQ ID NO: 106 - DNA, 30 bp, synthetic construct
  misc_feature 1..30: note = Probe sequence for bacterial rDNA 16s hypervariable region 3e
  source 1..30: mol_type = other DNA
tttttttcgttaccaacagaatactttttt

SEQ ID NO: 107 - DNA, 30 bp, synthetic construct
  misc_feature 1..30: note = Probe sequence for bacterial rDNA 16s hypervariable region 3e
  source 1..30: mol_type = other DNA
ttttttctacggtacctaatcagttttttt

SEQ ID NO: 108 - DNA, 30 bp, synthetic construct
  misc_feature 1..30: note = Probe sequence for bacterial rDNA 16s hypervariable region 3e
  source 1..30: mol_type = other DNA
ttttttctacggtaactaaccagttttttt

SEQ ID NO: 109 - DNA, 30 bp, synthetic construct
  misc_feature 1..30: note = Probe sequence for bacterial rDNA 16s hypervariable region 3e
  source 1..30: mol_type = other DNA
tttttttactttgaataagtgacgtttttt

SEQ ID NO: 110 - DNA, 30 bp, synthetic construct
  misc_feature 1..30: note = Probe sequence for bacterial rDNA 16s hypervariable region 3e
  source 1..30: mol_type = other DNA
tttttttggtaccttgtcagaaattttttt

SEQ ID NO: 111 - DNA, 30 bp, synthetic construct
  misc_feature 1..30: note = Probe sequence for bacterial rDNA 16s hypervariable region 3e
  source 1..30: mol_type = other DNA
ttttctactgtaccatttgaataatctttt

SEQ ID NO: 112 - DNA, 30 bp, synthetic construct
  misc_feature 1..30: note = Probe sequence for bacterial rDNA 16s hypervariable region 3g
  source 1..30: mol_type = other DNA
tttttttacctacgggaggcagcttttttt

SEQ ID NO: 113 - DNA, 30 bp, synthetic construct
  misc_feature 1..30: note = Probe sequence for bacterial rDNA 16s hypervariable region 3g
  source 1..30: mol_type = other DNA
ttttttctcctgcaggagacggtctttttt

SEQ ID NO: 114 - DNA, 30 bp, synthetic construct
  misc_feature 1..30: note = Probe sequence for bacterial rDNA 16s hypervariable region 6a
  source 1..30: mol_type = other DNA
tttttttcttaccaagtcttgactcttttt

SEQ ID NO: 115 - DNA, 30 bp, synthetic construct
  misc_feature 1..30: note = Probe sequence for bacterial rDNA 16s hypervariable region 6a
  source 1..30: mol_type = other DNA
tttttttcacctggtcttgacacttttttt

SEQ ID NO: 116 - DNA, 30 bp, synthetic construct
  misc_feature 1..30: note = Probe sequence for bacterial rDNA 16s hypervariable region 6a
  source 1..30: mol_type = other DNA
tttttctccttaccaggtcttgattttttt

SEQ ID NO: 117 - DNA, 30 bp, synthetic construct
  misc_feature 1..30: note = Probe sequence for bacterial rDNA 16s hypervariable region 6a
  source 1..30: mol_type = other DNA
tttttttccttacctactcttgtttttttt

SEQ ID NO: 118 - DNA, 30 bp, synthetic construct
  misc_feature 1..30: note = Probe sequence for bacterial rDNA 16s hypervariable region 6a
  source 1..30: mol_type = other DNA
tttttttctacctggccttgactctttttt

SEQ ID NO: 119 - DNA, 30 bp, synthetic construct
  misc_feature 1..30: note = Probe sequence for bacterial rDNA 16s hypervariable region 6a
  source 1..30: mol_type = other DNA
tttttttttaccaaatcttgacactttttt

SEQ ID NO: 120 - DNA, 30 bp, synthetic construct
  misc_feature 1..30: note = Probe sequence for bacterial rDNA 16s hypervariable region 6a
  source 1..30: mol_type = other DNA
tttttttcttacctagacttgactcttttt

SEQ ID NO: 121 - DNA, 30 bp, synthetic construct
  misc_feature 1..30: note = Probe sequence for bacterial rDNA 16s hypervariable region 6a
  source 1..30: mol_type = other DNA
tttttttaccttacccactcttgttttttt

SEQ ID NO: 122 - DNA, 30 bp, synthetic construct
  misc_feature 1..30: note = Probe sequence for bacterial rDNA 16s hypervariable region 6a
  source 1..30: mol_type = other DNA
ttttttccttacttaggtttgacacttttt

SEQ ID NO: 123 - DNA, 30 bp, synthetic construct
  misc_feature 1..30: note = Probe sequence for bacterial rDNA 16s hypervariable region 6c
  source 1..30: mol_type = other DNA
tttttctctttgaccactctagattttttt

SEQ ID NO: 124 - DNA, 30 bp, synthetic construct
  misc_feature 1..30: note = Probe sequence for bacterial rDNA 16s hypervariable region 6c
  source 1..30: mol_type = other DNA
tttttttatactagaaactttccatttttt

SEQ ID NO: 125 - DNA, 30 bp, synthetic construct
  misc_feature 1..30: note = Probe sequence for bacterial rDNA 16s hypervariable region 6c
  source 1..30: mol_type = other DNA
tttttttcccacggaagttttcattttttt

SEQ ID NO: 126 - DNA, 30 bp, synthetic construct
  misc_feature 1..30: note = Probe sequence for bacterial rDNA 16s hypervariable region 6c
  source 1..30: mol_type = other DNA
tttttttcccacagaactttcctttttttt

SEQ ID NO: 127 - DNA, 30 bp, synthetic construct
  misc_feature 1..30: note = Probe sequence for
  source 1..30: mol_type = other DNA
tttttttcccagagaacttagctttttttt

SEQ ID NO: 128 - DNA, 30 bp, synthetic construct
  misc_feature 1..30: note = Probe sequence for bacterial rDNA 16s hypervariable region 6c
  source 1..30: mol_type = other DNA
ttttttctcagcgaatcctttagttttttt

SEQ ID NO: 129 - DNA, 30 bp, synthetic construct
  misc_feature 1..30: note = Probe sequence for bacterial rDNA 16s hypervariable region 6c
  source 1..30: mol_type = other DNA
ttttttatcagagaatttagcagacttttt

SEQ ID NO: 130 - DNA, 30 bp, synthetic construct
  misc_feature 1..30: note = Probe sequence for bacterial rDNA 16s hypervariable region 6c
  source 1..30: mol_type = other DNA
ttttttttgctgagaactttcctttttttt

SEQ ID NO: 131 - DNA, 30 bp, synthetic construct
  misc_feature 1..30: note = Probe sequence for bacterial rDNA 16s hypervariable region 6c
  source 1..30: mol_type = other DNA
ttttttaatcctttgacaactctatttttt

SEQ ID NO: 132 - DNA, 30 bp, synthetic construct
  misc_feature 1..30: note = Probe sequence for bacterial rDNA 16s hypervariable region 6c
  source 1..30: mol_type = other DNA
ttcttaatcctttgaaaactctagttcttt

SEQ ID NO: 133 - DNA, 30 bp, synthetic construct
  misc_feature 1..30: note = Probe sequence for bacterial rDNA 16s hypervariable region 6c
  source 1..30: mol_type = other DNA
tttttttttccttctgaccggctctttttt

SEQ ID NO: 134 - DNA, 30 bp, synthetic construct
  misc_feature 1..30: note = Probe sequence for bacterial rDNA 16s hypervariable region 6c
  source 1..30: mol_type = other DNA
ttttttattccagagaactttccttttttt

SEQ ID NO: 135 - DNA, 30 bp, synthetic construct
  misc_feature 1..30: note = Probe sequence for bacterial rDNA 16s hypervariable region 6c
  source 1..30: mol_type = other DNA
ttttttttccttggcaaagttatgtttttt

SEQ ID NO: 136 - DNA, 30 bp, synthetic construct
  misc_feature 1..30: note = Probe sequence for bacterial rDNA 16s hypervariable region 6c
  source 1..30: mol_type = other DNA
ttttttttccttcgcaaagctatatttttt

SEQ ID NO: 137 - DNA, 30 bp, synthetic construct
  misc_feature 1..30: note = Probe sequence for bacterial rDNA 16s hypervariable region 6c
  source 1..30: mol_type = other DNA
tttttttcattgcgacgctatagttttttt

SEQ ID NO: 138 - DNA, 30 bp, synthetic construct
  misc_feature 1..30: note = Probe sequence for bacterial rDNA 16s hypervariable region 6c
  source 1..30: mol_type = other DNA
ttttttctttgcgatgctatagtttttttt

SEQ ID NO: 139 - DNA, 30 bp, synthetic construct
  misc_feature 1..30: note = Probe sequence for bacterial rDNA 16s hypervariable region 6c
  source 1..30: mol_type = other DNA
tttttttttccacagaacttagcttttttt

SEQ ID NO: 140 - DNA, 30 bp, synthetic construct
  misc_feature 1..30: note = Probe sequence for bacterial rDNA 16s hypervariable region 6c
  source 1..30: mol_type = other DNA
tttttttccctcggacgtttactttttttt

SEQ ID NO: 141 - DNA, 30 bp, synthetic construct
  misc_feature 1..30: note = Probe sequence for bacterial rDNA 16s hypervariable region 6e
  source 1..30: mol_type = other DNA
ttttttctgagctttcccttcgtttttttt

SEQ ID NO: 142 - DNA, 30 bp, synthetic construct
  misc_feature 1..30: note = Probe sequence for bacterial rDNA 16s hypervariable region 6e
  source 1..30: mol_type = other DNA
ttttttttgatgagaatgtgccactttttt

SEQ ID NO: 143 - DNA, 30 bp, synthetic construct
  misc_feature 1..30: note = Probe sequence for bacterial rDNA 16s hypervariable region 6e
  source 1..30: mol_type = other DNA
tttttttcagagcttccccttcgttttttt

SEQ ID NO: 144 - DNA, 30 bp, synthetic construct
  misc_feature 1..30: note = Probe sequence for bacterial rDNA 16s hypervariable region 6e
  source 1..30: mol_type = other DNA
ttttttttatgctttggtgccccttttttt

SEQ ID NO: 145 - DNA, 30 bp, synthetic construct
  misc_feature 1..30: note = Probe sequence for bacterial rDNA 16s hypervariable region 6e
  source 1..30: mol_type = other DNA
tttttttcatagaggagtgcctatcttttt

SEQ ID NO: 146 - DNA, 30 bp, synthetic construct
  misc_feature 1..30: note = Probe sequence for bacterial rDNA 16s hypervariable region 6e
  source 1..30: mol_type = other DNA
tttttttatgctttagtgcctttttttttt

SEQ ID NO: 147 - DNA, 30 bp, synthetic construct
  misc_feature 1..30: note = Probe sequence for bacterial rDNA 16s hypervariable region 6e
  source 1..30: mol_type = other DNA
tttttttttgatggattggtgctctttttt

SEQ ID NO: 148 - DNA, 30 bp, synthetic construct
  misc_feature 1..30: note = Probe sequence for bacterial rDNA 16s hypervariable region 6e
  source 1..30: mol_type = other DNA
ttttcttatagagccttccccttctttttt

SEQ ID NO: 149 - DNA, 30 bp, synthetic construct
  misc_feature 1..30: note = Probe sequence for bacterial rDNA 16s hypervariable region 6e
  source 1..30: mol_type = other DNA
ttttttttaggctttctcttcgtttttttt

SEQ ID NO: 150 - DNA, 30 bp, synthetic construct
  misc_feature 1..30: note = Probe sequence for bacterial rDNA 16s hypervariable region 6e
  source 1..30: mol_type = other DNA
ttttttcaaacataatggaggttctttttt

SEQ ID NO: 151 - DNA, 30 bp, synthetic construct
  misc_feature 1..30: note = Probe sequence for bacterial rDNA 16s hypervariable region 6e
  source 1..30: mol_type = other DNA
tttttttagatatagtggaggttctttttt

SEQ ID NO: 152 - DNA, 30 bp, synthetic construct
  misc_feature 1..30: note = Probe sequence for bacterial rDNA 16s hypervariable region 6e
  source 1..30: mol_type = other DNA
tttttaaatatagttgaggttaatattttt

SEQ ID NO: 153 - DNA, 30 bp, synthetic construct
  misc_feature 1..30: note = Probe sequence for bacterial rDNA 16s hypervariable region 6g
  source 1..30: mol_type = other DNA
tttttttggacaaagtgacaggctattttt

SEQ ID NO: 154 - DNA, 30 bp, synthetic construct
  misc_feature 1..30: note = Probe sequence for bacterial rDNA 16s hypervariable region 6g
  source 1..30: mol_type = other DNA
tttttttggaatctagatacaggctttttt

SEQ ID NO: 155 - DNA, 30 bp, synthetic construct
  misc_feature 1..30: note = Probe sequence for bacterial rDNA 16s hypervariable region 6g
  source 1..30: mol_type = other DNA
tttttattggaaccgtgagacttttttttt

SEQ ID NO: 156 - DNA, 30 bp, synthetic construct
  misc_feature 1..30: note = Probe sequence for bacterial rDNA 16s hypervariable region 6g
  source 1..30: mol_type = other DNA
tttttttggcaaagtgacaggctatttttt

SEQ ID NO: 157 - DNA, 30 bp, synthetic construct
  misc_feature 1..30: note = Probe sequence for bacterial rDNA 16s hypervariable region 6g
  source 1..30: mol_type = other DNA
ttttttttggaactctgagacatttttttt

SEQ ID NO: 158 - DNA, 30 bp, synthetic construct
  misc_feature 1..30: note = Probe sequence for bacterial rDNA 16s hypervariable region 6g
  source 1..30: mol_type = other DNA
ttttttattgggaacgctgagatttttttt

SEQ ID NO: 159 - DNA, 30 bp, synthetic construct
  misc_feature 1..30: note = Probe sequence for bacterial rDNA 16s hypervariable region 6g
  source 1..30: mol_type = other DNA
ttttttttttcgggagctctgatttttttt

SEQ ID NO: 160 - DNA, 30 bp, synthetic construct
  misc_feature 1..30: note = Probe sequence for bacterial rDNA 16s hypervariable region 6g
  source 1..30: mol_type = other DNA
tttttttgaacagagacacaggtttttttt

SEQ ID NO: 161 - DNA, 30 bp, synthetic construct
  misc_feature 1..30: note = Probe sequence for bacterial rDNA 16s hypervariable region 6g
  source 1..30: mol_type = other DNA
tttttttgaactcagacacaggcttttttt

SEQ ID NO: 162 - DNA, 30 bp, synthetic construct
  misc_feature 1..30: note = Probe sequence for bacterial rDNA A16s hypervariable region 6g
  source 1..30: mol_type = other DNA
tttttttagcagaagtgacagtcttttttt

SEQ ID NO: 163 - DNA, 30 bp, synthetic construct
  misc_feature 1..30: note = Probe sequence for bacterial rDNA 16s hypervariable region 6g
  source 1..30: mol_type = other DNA
ttttttttaggttaaccgagtgtttttttt

SEQ ID NO: 164 - DNA, 30 bp, synthetic construct
  misc_feature 1..30: note = Probe sequence for bacterial rDNA 16s hypervariable region 6g
  source 1..30: mol_type = other DNA
ttttttttaggttatcggagtgttattttt

SEQ ID NO: 165 - DNA, 30 bp, synthetic construct
  misc_feature 1..30: note = Probe sequence for bacterial rDNA 16s hypervariable region 6g
  source 1..30: mol_type = other DNA
tttttttggttaacaatatgacattttttt

SEQ ID NO: 166 - DNA, 30 bp, synthetic construct
  misc_feature 1..30: note = Probe sequence for bacterial rDNA 16s hypervariable region 6g
  source 1..30: mol_type = other DNA
tttttattggaactgtgagacatttttttt

SEQ ID NO: 167 - DNA, 32 bp, synthetic construct
  misc_feature 1..32: note = Probe sequence for fungal rDNA 28s D2 region 1a
  source 1..32: mol_type = other DNA
ttttttcggccagcatcagtttggtctttttt

SEQ ID NO: 168 - DNA, 32 bp, synthetic construct
  misc_feature 1..32: note = Probe sequence for fungal rDNA 28s D2 region 1b
  source 1..32: mol_type = other DNA
ttttttttcacggcttctgctgtgcttttttt

SEQ ID NO: 169 - DNA, 32 bp, synthetic construct
  misc_feature 1..32: note = Probe sequence for fungal rDNA 28s D2 region 1b
  source 1..32: mol_type = other DNA
ttttttttctctgcgcctcggtgttttttttt

SEQ ID NO: 170 - DNA, 32 bp, synthetic construct
  misc_feature 1..32: note = Probe sequence for negative control
  source 1..32: mol_type = other DNA
tttttctactacctatgctgattcactctttt

SEQ ID NO: 171 - DNA, 30 bp, synthetic construct
  misc_feature 1..30: note = Probe sequence for RNaseP positive control
  source 1..30: mol_type = other DNA
tttttctgacctgaaggctctgcgcttttt

SEQ ID NO: 172 - DNA, 30 bp, synthetic construct
  misc_feature 1..30: note = Probe sequence for RNaseP control fragment
  source 1..30: mol_type = other DNA
tttttctgtccagatggcacagcgcttttt

SEQ ID NO: 173 - DNA, 790 bp, synthetic construct
  misc_feature 1..790: note = Probe sequence for 16s quantitation reference standard
  source 1..790: mol_type = other DNA
tctgagaggatgaccagccacactggaactgagacacggtccagtttcctgcaggagacggttgtggggaatattgcacaatgggcgcaagcctgatgcagccatgccgcgtgtatgaagaatgccttcgggtagtaaagtactttcagtggggaggatgtgagttaagttactaccttagctcatagaccttaccagcagtagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaacctaacctgctctagacatcctcggacgtttacagagctgagactgtgacttcgagaacagtgagtcaggtgctgcatggctgtcgtcagctcgtgttgtgaaat

SEQ ID NO: 174 - DNA, 17 bp, synthetic construct
  misc_feature 1..17: note = Probe sequence for Escherichia coli 16s hypervariable region 3a
  source 1..17: mol_type = other DNA
aagggagtaaagttaat

SEQ ID NO: 175 - DNA, 16 bp, synthetic construct
  misc_feature 1..16: note = Probe sequence for Escherichia coli 16s hypervariable region 3c
  source 1..16: mol_type = other DNA
tacctttgctcattga

SEQ ID NO: 176 - DNA, 14 bp, synthetic construct
  misc_feature 1..14: note = Probe sequence for Escherichia coli 16s hypervariable region 3e
  source 1..14: mol_type = other DNA
acgttacccgcaga

SEQ ID NO: 177 - DNA, 59 bp, synthetic construct
  misc_feature 1..59: note = Speciation probe sequence for Escherichia coli
  source 1..59: mol_type = other DNA
aggaagggagtaaagttaatacctttgctcattgacgttacccgcagaagaagcaccgg

SEQ ID NO: 178 - DNA, 59 bp, synthetic construct
  misc_feature 1..59: note = Speciation probe sequence for Enterobacter cloacae
  source 1..59: mol_type = other DNA
aggaaggtgttgtggttaataaccgcagcaattgacgttacccgcagaagaagcaccgg

SEQ ID NO: 179 - DNA, 59 bp, synthetic construct
  misc_feature 1..59: note = Speciation probe sequence for Serratia marcesans
  source 1..59: mol_type = other DNA
aggaaggtggtgagcttaatacgttcatcaattgacgttactcgcagaagaagcaccgg

SEQ ID NO: 180 - DNA, 59 bp, synthetic construct
  misc_feature 1..59: note = Speciation probe sequence for Citrobacter braakii
  source 1..59: mol_type = other DNA
aggaaggtgttgtggttaataaccgcagcaattgacgttactcgcagaagaagcaccgg

SEQ ID NO: 181 - DNA, 59 bp, synthetic construct
  misc_feature 1..59: note = Speciation probe sequence for Citrobacter freundii
  source 1..59: mol_type = other DNA
aggaaggcgttgtggttaataaccgcagcgattgacgttactcgcagaagaagcaccgg

SEQ ID NO: 182 - DNA, 59 bp, synthetic construct
  misc_feature 1..59: note = Speciation probe sequence for Staphylococcus aureus
  source 1..59: mol_type = other DNA
aagaacatatgtgtaagtaactgtgcacatcttgacggtacctaatcagaaagccacgg

SEQ ID NO: 183 - DNA, 59 bp, synthetic construct
  misc_feature 1..59: note = Speciation probe sequence for Staphylococcus saprophyticus
  source 1..59: mol_type = other DNA
aagaacaaacgtgtaagtaactgtgcacgtcttgacggtacctaatcagaaagccacgg

SEQ ID NO: 184 - DNA, 59 bp, synthetic construct
  misc_feature 1..59: note = Speciation probe sequence for Staphylococcus epidermidis
  source 1..59: mol_type = other DNA
aagaacaaatgtgtaagtaactatgcacgtcttgacggtacctaatcagaaagccacgg

SEQ ID NO: 185 - DNA, 17 bp, synthetic construct
  misc_feature 1..17: note = Probe sequence for Candida albicans 28s region 1a
  source 1..17: mol_type = other DNA
ggccagcatcggtttgg

SEQ ID NO: 186 - DNA, 20 bp, synthetic construct
  misc_feature 1..20: note = Probe sequence for Candida albicans 28s region 1b
  source 1..20: mol_type = other DNA
cacggcttctgctgtgtgtt

SEQ ID NO: 187 - DNA, 66 bp, synthetic construct
  misc_feature 1..66: note = Speciation probe sequence for Candida glabrata
  source 1..66: mol_type = other DNA
ggccagcatcggtttggagcggcaggataatggcggaggaatgtggcacggcttctgctgtgtgtt

SEQ ID NO: 188 - DNA, 68 bp, synthetic construct
  misc_feature 1..68: note = Speciation probe sequence for Candida glabrata
  source 1..68: mol_type = other DNA
ggccagcatcggttttggcggccggaaaaacctagggaatgtggctctgcgcctcggtgtagagtgtt

SEQ ID NO: 189 - DNA, 66 bp, synthetic construct
  misc_feature 1..66: note = Speciation probe sequence for Candida dubliniensis
  source 1..66: mol_type = other DNA
ggccagcatcggtttggagcggtaggataatggcgggggaatgtggcacgactttggttgtctgtt

SEQ ID NO: 190 - DNA, 65 bp, synthetic construct
  misc_feature 1..65: note = Speciation probe sequence for Candida tropicalis
  source 1..65: mol_type = other DNA
ggccagcatcagtttgggcggtaggagaattgcgttggaatgtggcacggcttcggttgtgtgtt

SEQ ID NO: 191 - DNA, 65 bp, synthetic construct
  misc_feature 1..65: note = Speciation probe sequence for Candida orthopsilosis
  source 1..65: mol_type = other DNA
ggccagcatcagtttgggcggtaggacaattgcaaagaaatgtggcactgcctcggtagtgtgtt

SEQ ID NO: 192 - DNA, 65 bp, synthetic construct
  misc_feature 1..65: note = Speciation probe sequence for Candida parapsilosis
  source 1..65: mol_type = other DNA
ggccagcatcagtttgagcggtaggataagtgcaaagaaatgtggcactgcttcggtagtgtgtt

SEQ ID NO: 193 - DNA, 42 bp, synthetic construct
  misc_feature 1..42: note = Forward primer sequence for rDNA 16s hypervariable region 3
  source 1..42: mol_type = other DNA
tttctaccgtactctagctccatgccgcgtgwgtgaagaagg

SEQ ID NO: 194 - DNA, 42 bp, synthetic construct
  misc_feature 1..42: note = Forward primer sequence for rDNA 16s hypervariable region 3
  source 1..42: mol_type = other DNA
tttctaccgtactctagctcaacgccgcgtgagtgatgaagg

SEQ ID NO: 195 - DNA, 34 bp, synthetic construct
  misc_feature 1..34: note = Reverse primer sequence for rDNA 16s hypervariable region 3
  source 1..34: mol_type = other DNA
ataactcaacagtgtattaccgcggctgctggca

SEQ ID NO: 196 - DNA, 39 bp, synthetic construct
  misc_feature 1..39: note = Forward primer sequence for rDNA 16s hypervariable region 3
  source 1..39: mol_type = other DNA
tttctaccgtactctagctgagcatgtggtttaatycga

SEQ ID NO: 197 - DNA, 55 bp, synthetic construct
  misc_feature 1..55: note = Reverse primer sequence for rDNA 16s hypervariable region 3
  source 1..55: mol_type = other DNA
aatttaatacgactcactatagggataactcaacagagctgacgacayccatgca

SEQ ID NO: 198 - DNA, 17 bp, synthetic construct
  misc_feature 1..17: note = Forward primer detector sequence
  source 1..17: mol_type = other DNA
tctaccgtactctagct

SEQ ID NO: 199 - DNA, 15 bp, synthetic construct
  misc_feature 1..15: note = T7 detector sequence
  source 1..15: mol_type = other DNA
ctgttgagttatcct

SEQ ID NO: 200 - DNA, 17 bp, synthetic construct
  misc_feature 1..17: note = Probe sequence for 16s hypervariable region 3c in Enterococcus faecium
  source 1..17: mol_type = other DNA
taactgttcatcccttg

SEQ ID NO: 201 - DNA, 15 bp, synthetic construct
  misc_feature 1..15: note = Probe sequence for 16s hypervariable region 3c in Staphylococcus aureus
  source 1..15: mol_type = other DNA
tgtgcacatcttgac

SEQ ID NO: 202 - DNA, 14 bp, synthetic construct
  misc_feature 1..14: note = Probe sequence for 16s hypervariable region 6c in Klebsiella pneumonia
  source 1..14: mol_type = other DNA
ccacagaactttcc

SEQ ID NO: 203 - DNA, 17 bp, synthetic construct
  misc_feature 1..17: note = Probe sequence for 16s hypervariable region 3a in Acinetobacter baumannii
  source 1..17: mol_type = other DNA
gctactttagttaatac

SEQ ID NO: 204 - DNA, 16 bp, synthetic construct
  misc_feature 1..16: note = Probe sequence for 16s hypervariable region 3e in Pseudomonas aeruginosa
  source 1..16: mol_type = other DNA
cgttaccaacagaata

SEQ ID NO: 205 - DNA, 15 bp, synthetic construct
  misc_feature 1..15: note = Probe sequence for 16s hypervariable region 3a in Enterobacter cloacae
  source 1..15: mol_type = other DNA
aaggtgttgtggtta

SEQ ID NO: 206 - DNA, 14 bp, synthetic construct
  misc_feature 1..14: note = Probe sequence for 16s hypervariable region 6c in Klebsiella aerogenes
  source 1..14: mol_type = other DNA
ccagagaacttagc

SEQ ID NO: 207 - DNA, 16 bp, synthetic construct
  misc_feature 1..16: note = Probe sequence for 16s hypervariable region 3c in Escherichia coli
  source 1..16: mol_type = other DNA
tacctttgctcattga

(19) United States
(12) Patent Application Publication
(10) Pub. No.: US 2025/0092470 A1
(43) Pub. Date: Mar. 20, 2025

(54) Methods for Detecting and Discriminating among Microbes in Microbial Pathogen Mixtures

(71) Applicant: PathogenDx, Inc., Scottsdale, AZ (US)

(72) Inventors: Michael E. Hogan, Stony Brook, NY (US); Frederick H. Eggers, Sahuarita, AZ (US); Melissa R. May, Tucson, AZ (US); Fushi Wen, Tucson, AZ (US); Kevin O'Brien, Sahuarita, AZ (US); Benjamin A. Katchman, Tucson, AZ (US)

(73) Assignee: PathogenDx, Inc., Scottsdale, AZ (US)

(21) Appl. No.: 18/891,871

(22) Filed: Sep. 20, 2024

Related U.S. Application Data
(60) Provisional application No. 63/584,008, filed on Sep. 20, 2023.

(51) Int. Cl. C12Q 1/689; C12Q 1/6818; C12Q 1/6895; G01N 21/64

(52) U.S. Cl. CPC C12Q 1/689; C12Q 1/6818; C12Q 1/6895; G01N 21/6428; G01N 21/6458; C12Q 2600/156; C12Q 2600/16; C12Q 2600/166; G01N 2021/6439

(57) ABSTRACT

Provided herein are methods for detecting and discriminating bacteria and fungal species by nucleic acid amplification, then hybridization analysis on a microarray. Primer pairs are used to simultaneously amplify multiple ribosomal Hypervariable regions from the rDNA or rRNA in bacteria and/or fungi as a multiplex amplification reaction, wherein the resulting hypervariable region amplicon mix is analyzed by hybridization to nucleic acid probes that are complementary to unique sequence determinants within those amplified hypervariable regions. The combination of multiplex amplification and microarray hybridization is used for detection and differentiation of specific species within a microbial set relevant to human or animal health or environmental or agricultural analysis, based on simultaneous analysis of the sequence in two or more hypervariable regions, concurrently.

### CROSS-REFERENCE TO RELATED APPLICATIONS

This non-provisional patent application claims benefit under 35 U.S.C. § 119 (e) of provisional application U.S. Ser. No. 63/584,008, filed Sep. 20, 2023, the entirety of which is hereby incorporated by reference.

### SEQUENCE LISTING

A sequence listing is electronically submitted in XML format in compliance with 37 C.F.R. § 1.831 (a) and is incorporated by reference herein. The XML file is named D7962SEQ, was created on Sep. 18, 2024 and is 267 KB in size.

### BACKGROUND OF THE INVENTION

### Field of the Invention

The present invention relates to the field of identification of pathogenic bacteria and pathogenic fungal. More particularly, the present invention relates to methods to detect and to discriminate among a set of bacteria and fungi via microarray analysis.

### Description of the Related Art

Bacteria and fungi are a major cause of disease in humans and animals especially infections of urine, blood, or wounds. Such infection can manifest symptomatically as a “syndrome” which may be caused by any one of or combinations among a set of bacteria or fungal pathogens. The early detection of the disease-causing pathogen or pathogens in such syndromic presentation requires the ability to detect and discriminate multiple candidate microbial species quickly and simultaneously among a much larger set of microbes that can produce an infection in the blood or urine or a wound. Such testing is often referred to as “Syndromic Testing for Infectious Disease” that use multiplex methods for testing a large set of pathogens in parallel, which alone or in combination could be linked to a syndrome such as urinary tract infection or blood infection or wound infection. Generally, such a syndromic pathogen set may comprise bacteria, fungi or viruses and the syndromic testing could be based on nucleic acid or protein analytes. The present invention is focused on nucleic acid testing only and to detect and to discriminate causative bacteria and fungal pathogens.

Historically, such syndromic infections have been interrogated by culture methods, often requiring multiple days of culture testing on multiple growth media to detect and discriminate the causative pathogens. In some cases, such pathogens of concern culture slowly or are hard to differentiate from other possible members of a syndromic test panel. Thus, rapid screening of a sample to detect and discriminate one or combinations of microbial contamination, without culture, in hours rather than days could save lives by enabling early identification of the specific combination of microbes presenting in a subject, to guide diagnosis and treatment.

Thus, the prior art is deficient in means and methods of fast, culture-free detection and differentiation of multiple bacterial and fungal species in a single assay. Specifically, the prior art is deficient in methods that enable discrimination among elements of a syndromic test panel comprising multiple candidate bacteria and fungi as a high throughput lab-based molecular test.

### SUMMARY OF THE INVENTION

The present invention is directed to a method for method for detecting and discriminating among multiple bacterial species and/or fungal species in a sample. In the method a sample is obtained from a subject. Bacteria and fungi are harvested from the sample and nucleic acids are isolated from the harvested bacteria and fungi. The bacterial and/or fungal ribosomal DNA or ribosomal RNA are amplified using at least two fluorescently-labeled primer pairs selective for bacterial and fungal hypervariable regions to generate at least two fluorescently labeled ribosomal nucleic acid amplicons. The fluorescently-labeled ribosomal nucleic acid amplicons are hybridized to a plurality of nucleic acid probes, where each probe in the plurality has a sequence complementary to a sequence determinant in one of the amplified ribosomal nucleic acid hypervariable regions and each probe is attached to a microarray. The microarray is washed at least once and imaged to detect a fluorescent signal from each of the two or more fluorescently-labeled ribosomal gene amplicons hybridized to the complementary probe. At least two hypervariable regions are analyzed concurrently, thereby detecting the bacterial species and/or fungal species in the sample.

The present invention also is directed to a related method that further comprises amplifying a region of interest in at least one additional gene using at least one fluorescently-labeled primer pair selective for the region of interest to generate at least one additional fluorescently labeled amplicon. The at least one additional fluorescently labeled amplicon is hybridized where the plurality of nucleic acid probes comprising the at least one additional probe has a sequence complementary to the region of interest. The microarray is imaged to detect a fluorescent signal from each of the at least one additional fluorescently-labeled amplicons hybridized to the complementary probe.

The present invention is directed further to a related method that further comprises quantifying an abundance of each species in the sample. In the method a known number of copies of a quantitative reference standard is added to the amplifying step where the quantitative reference standard has a nucleotide sequence similar to the nucleotide sequences of the hypervariable regions that are amplified. A probe with a sequence complementary to the nucleotide sequence of the quantitative reference standard is added to the microarray and the quantitative reference standard is hybridized to the complementary probe. A ratio of the hybridization signal in relative fluorescence units from the fluorescently-labeled amplicons hybridized to their complementary probes to a hybridization signal in relative fluorescence units from the quantitative reference standard hybridized to its complementary probe is measured. The ratio is uniquely correlated to a gene copy number of bacterial species or fungal species in the sample relative to the number of copies of the quantitative reference standard.

These and other features, aspects, and advantages of the embodiments of the present disclosure will become better understood when the following detailed description is read with reference to the accompanying drawings.

### BRIEF DESCRIPTION OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIG. 1 shows the structure of ribosomal hypervariable regions in bacteria.

FIG. 2 shows the predicted pattern of hybridization of bacterial and fungal species to a representative panel of species-specific probes

FIG. 3 is a design of a manufactured microarray test (1313) containing selected hypervariable probe/species to test for a urinary tract infection (UTI).

FIGS. 4A-4B illustrate the automated microarray 4 hr workflow for a UTI-D³™ Array (FIG. 4A) and identify the speciation and controls utilized (FIG. 4B).

FIGS. 5A-5C show experimental hybridization specificity data for 1313 UTI array, based on purified gDNA in an HV1a or 1c probe grid (FIG. 5A), an HV3a & 3c grid (FIG. 5B) and an HV6 c,e probe grid (FIG. 5C).

FIG. 6 shows the experimental hybridization limit of detection and specificity data for the 1313 array for purified gDNA.

FIGS. 7A-7C show the structure of ribosomal 16S hypervariable regions in bacteria (FIG. 7A) and 28S hypervariable regions in fungi (FIG. 7B) as an extension of the graphical layout in FIG. 1 and identification of the sequences (FIG. 7C).

FIGS. 8A-8B show the predicted pattern of hybridization of bacterial and fungal species to a representative panel of species-specific probes with addition of new species targets (bold italic) relative to that in FIG. 2.

FIGS. 9A-9B show a design of a manufactured microarray test (1319) containing selected multiple hypervariable probe/species to test for a urinary tract infection (UTI) with addition of new content specified by FIGS. 8A-8B

FIGS. 10A-10B show the experimental hybridization limit of detection and specificity data for the 1319 array for purified gDNA based on PCR amplification of 16S-HV3 and HV6 regions only and hybridization to HV3a,c and HV6 probes (FIG. 10A). Updated Specificity data (HV3 and HV6) have been presented as a matrix as in FIGS. 5A-5B, where perfect specificity would generate only positive species determination on diagonal elements (FIG. 10B). Updated LOD data have been presented in the same format as in FIG. 6.

FIGS. 11A-11C show the quantitation of multiple bacterial species by reference to internal standards.

FIGS. 12A-12B show the use of RT-PCR rather than PCR to Detect and Discriminate Species, based on concordant analysis of multiple hypervariable regions of mature ribosomal RNA transcripts (rRNA) obtained from ribosomes rather than ribosomal DNA (rDNA) obtained from genomic DNA.

FIGS. 13A-13H show the isothermal NASBA amplification and D3 Array-UTI detection of the eight ESKAPE++pathogens.

### DETAILED DESCRIPTION OF THE INVENTION

The articles “a” and “an” when used in conjunction with the term “comprising” in the claims and/or the specification, may refer to “one”, but it is also consistent with the meaning of “one or more”, “at least one”, and “one or more than one”. Some embodiments of the invention may consist of or consist essentially of one or more elements, components, method steps, and/or methods of the invention. It is contemplated that any composition, component or method described herein can be implemented with respect to any other composition, component or method described herein.

The term “or” in the claims refers to “and/or” unless explicitly indicated to refer to alternatives only or the alternatives are mutually exclusive, although the disclosure supports a definition that refers to only alternatives and “and/or”.

The terms “comprise” and “comprising” are used in the inclusive, open sense, meaning that additional elements may be included.

The terms “consist of” and “consisting of” are used in the exclusive, closed sense, meaning that additional elements may not be included.

The term “including” is used herein to mean “including, but not limited to”. “Including” and “including but not limited to” are used interchangeably.

As used herein, the term “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

As used herein, the terms “microarray” and “microarray support” are interchangeable.

As used herein, the term “subject” refers to a human subject or other mammal.

As used herein, the term “ESKAPE++pathogens” refers to the six pathogens Enterococcus faecium, Staphylococcus aureus, Klebsiella pneumoniae, Acinetobacter baumannii, Pseudomonas aeruginosa, and Enterobacter spp., for example Enterobacter cloacae, plus the two additional pathogens Escherichia coli and Klebsiella aerogenes that are characterized by having increased levels of resistance to multiple classes of antibiotics.

In one embodiment of this invention, there is provided a method for detecting and discriminating among multiple bacterial species and fungal species in a sample, comprising obtaining a sample from a subject; harvesting bacteria and fungi from the sample; isolating nucleic acids from the harvested bacteria and fungi; amplifying bacterial and/or fungal ribosomal DNA or ribosomal RNA using at least two fluorescently-labeled primer pairs selective for bacterial and fungal hypervariable regions to generate at least two fluorescently labeled ribosomal nucleic acid amplicons; hybridizing the fluorescently-labeled ribosomal nucleic acid amplicons to a plurality of nucleic acid probes, each probe in the plurality having a sequence complementary to a sequence determinant in one of the amplified ribosomal nucleic acid hypervariable regions and each probe attached to a microarray; washing the microarray at least once; and imaging the microarray to detect a fluorescent signal from each of the two or more fluorescently-labeled ribosomal gene amplicons hybridized to the complementary probe; and analyzing concurrently at least two hypervariable regions, thereby detecting the bacterial species and/or fungal species in the sample. In this embodiment the amplifying step may comprise a PCR amplification, an RT-PCR amplification or an isothermal Transcription Mediated NASBA Amplification of rRNA.

Further to this embodiment the method comprises amplifying a region of interest in at least one additional gene using at least one fluorescently-labeled primer pair selective for the region of interest to generate at least one additional fluorescently labeled amplicon; hybridizing the at least one additional fluorescently labeled amplicon, where the plurality of nucleic acid probes comprises at least one additional probe having a sequence complementary to the region of interest; and imaging the microarray to detect a fluorescent signal from each of the at least one additional fluorescently-labeled amplicons hybridized to the complementary probe. In one aspect of both embodiments one of the at least one additional genes may comprise human RNaseP as an internal standard. In this aspect the fluorescently-labeled primer pair for the human RNaseP gene may have a nucleotide sequence of SEQ ID NOS: 10-11. In this aspect, the human RNaseP probe may have a nucleic acid of SEQ ID NO: 171. In another aspect of both embodiments, the at least one additional genes may comprise genes that confer antibiotic resistance to the bacterial species. In this aspect the antibiotic resistant bacterial species may be Enterococcus faecium, Staphylococcus aureus, Klebsiella pneumoniae, Acinetobacter baumannii, Pseudomonas aeruginosa, Enterobacter cloacae, Escherichia coli and Klebsiella aerogenes.

In another further embodiment, the method comprises a step of quantifying an abundance of each species in the sample. In this further embodiment, the method may comprise adding a known number of copies of a quantitative reference standard to the amplifying step wherein said quantitative reference standard has a nucleotide sequence similar to the nucleotide sequences of the hypervariable regions that are amplified; adding to the microarray a probe with a sequence complementary to the nucleotide sequence of the quantitative reference standard; hybridizing the quantitative reference standard to the complementary probe; measuring a ratio of the hybridization signal in relative fluorescence units from the fluorescently-labeled amplicons hybridized to their complementary probes to a hybridization signal in relative fluorescence units from the quantitative reference standard hybridized to its complementary probe; and correlating uniquely the ratio to a gene copy number of bacterial species or fungal species in the sample relative to the number of copies of the quantitative reference standard. In this further embodiment, the quantitative reference standard may have a probe nucleotide sequence of SEQ ID NO: 173.

In all embodiments and aspects thereof, the fluorescently-labeled primer pair comprises a pair of nucleotide sequences that targets a bacterial 16S rDNA hypervariable region or a 28S D2 region, said fluorescently-labeled primer pair selected from the group consisting of SEQ ID NOS: 1 and 2, SEQ ID NOS: 1 and 3, SEQ ID NOS: 4 and 5, SEQ ID NOS: 6 and 7, SEQ ID NOS: 8 and 9 and a combination thereof.

In all embodiments and aspects thereof, the plurality of hybridization probes may comprise a set of bacterial probes each with a nucleotide sequence corresponding to the sequence determinant in a 16S hypervariable region 1 selected from the group consisting of SEQ ID NOS: 12-31, SEQ ID NOS: 32-50 and a combination thereof. Further to this, the plurality of hybridization probes may comprise a set of bacterial probes each with a nucleotide sequence corresponding to the sequence determinant in a 16S hypervariable region 3 selected from the group consisting of SEQ ID NOS: 51-75, SEQ ID NOS: 76-100, SEQ ID NOS: 101-111, SEQ ID NOS: 112-113 and a combination thereof. Further yet the plurality of hybridization probes may comprise a set of bacterial probes each with a nucleotide sequence corresponding to the sequence determinant in a 16S hypervariable region 6 selected from the group consisting of SEQ ID NOS: 114-122, SEQ ID NOS: 123-140, SEQ ID NOS: 141-152, SEQ ID NOS: 153-166 and a combination thereof. Further yet the plurality of hybridization probes may comprise a set of bacterial probes each with a nucleotide sequence corresponding to the sequence determinant in a 28S D2 region selected from the group consisting of SEQ ID NO: 167, SEQ ID NOS: 168-169 and a combination thereof.

In all embodiments and aspects thereof, the bacteria and the fungi may be associated with detecting and treating a urinary tract infection, a blood infection or a wound infection. Particularly, the bacteria associated with the urinary tract infection may be Acinetobacter baumannii, Aerococcus urinae, Citrobacter freundii, Citrobacter koseri, Enterobacter aerogenes, Enterobacter cloacae, Enterococcus faecalis, Enterococcus faecium, Escherichia coli, Klebsiella oxytoca, Klebsiella pneumoniae, Morganella morganii, Mycoplasma hominis, Mycoplasma genitalium, Proteus mirabilis, Proteus vulgaris, Providencia stuartii, Pseudomonas aeruginosa, Serratia marcescens, Staphylococcus aureus, Staphylococcus saprophyticus, Streptococcus agalactiae, Ureaplasma parvum, or Ureaplasma urealyticum and the fungi associated with the urinary tract infection are Candida albicans or Candida glabrata. In addition, the subject may be a human or other mammal or a plant. Furthermore, the sample is a blood sample, a urine sample, a wound swab, a urogenital swab or other equivalent sample type, an air sample, a water sample, or a surface swab.

Provided herein are methods for detecting and discriminating among multiple bacterial species and fungal species in a sample. In the method, a sample is obtained from a subject, Bacteria and fungi are harvested from a sample obtained from a subject and nucleic acids are isolated therefrom. An amplification reaction is performed on the Bacterial and/or fungal ribosomal DNA or ribosomal RNA are amplified using at least two fluorescently-labeled primer pairs selective for two or more hypervariable regions of ribosomal genes or the RNA product of such genes. Two or more fluorescently-labeled ribosomal DNA or ribosomal RNA amplicons are generated thereby.

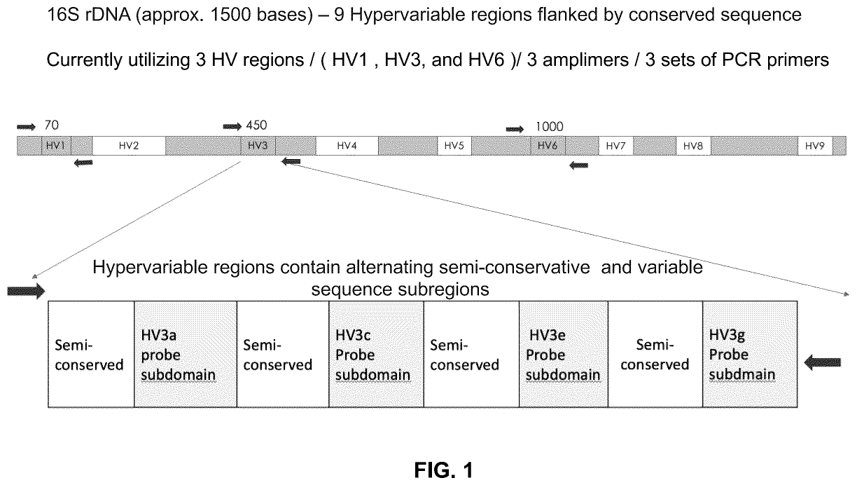

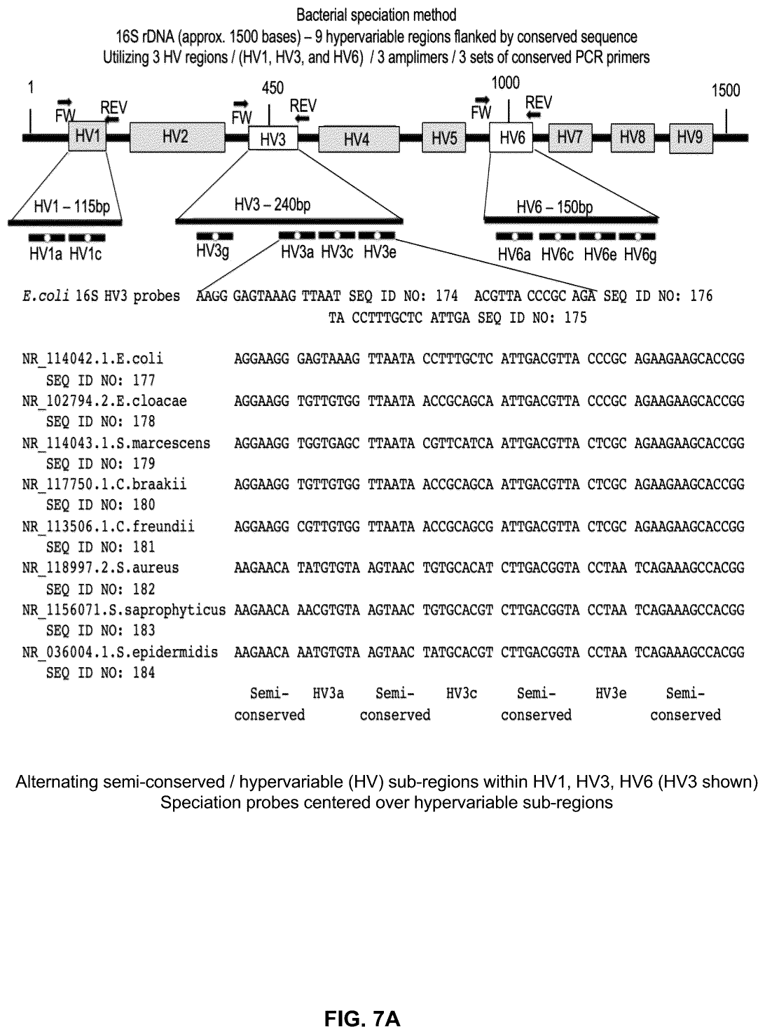

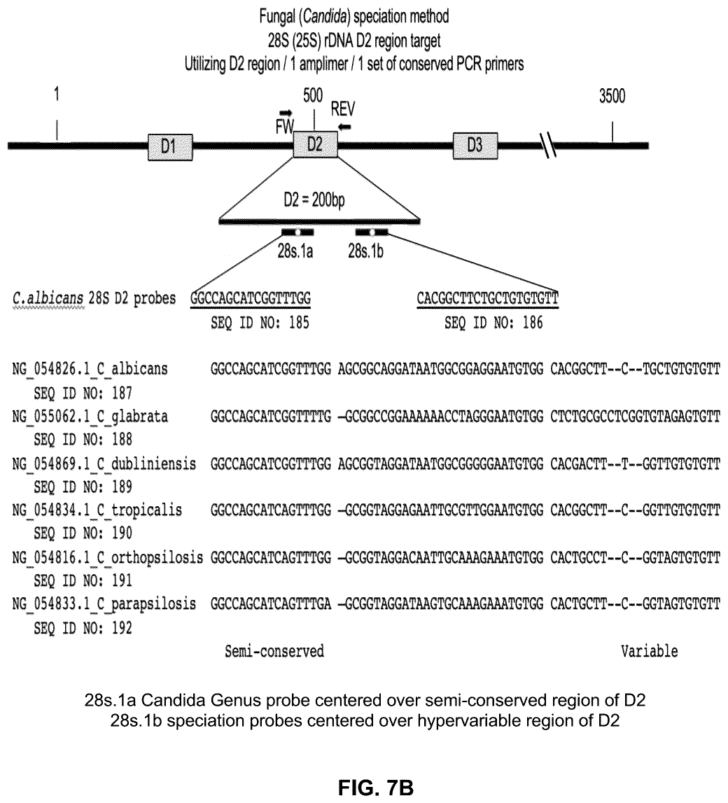

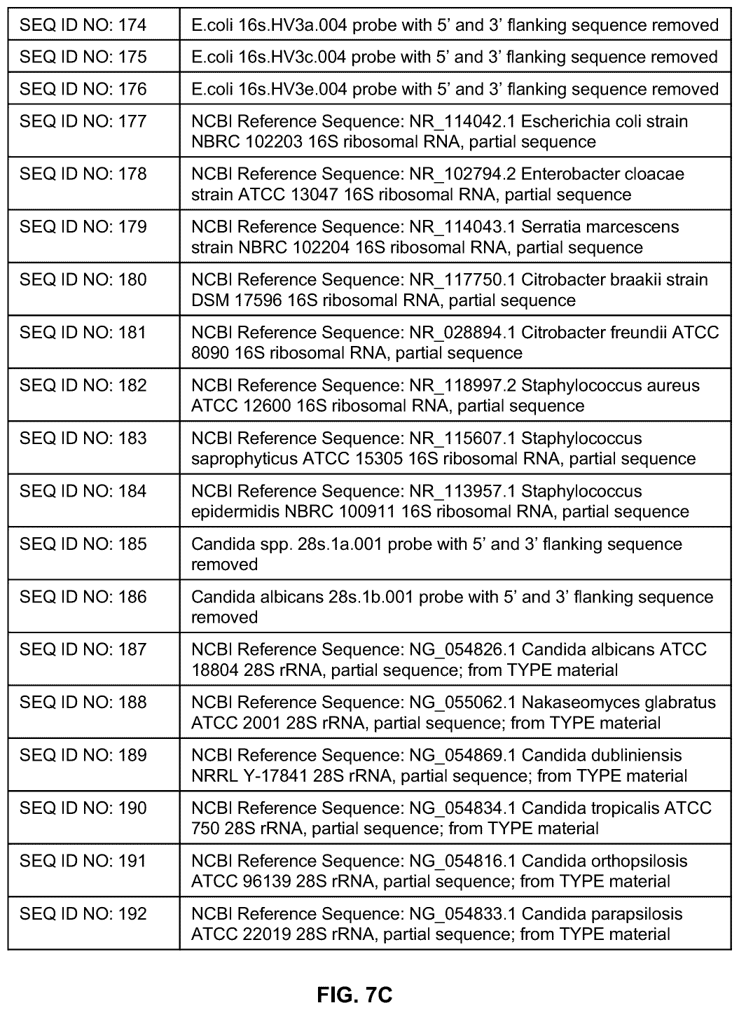

Such hypervariable (HV) regions of the ribosomal genes (rDNA) and the rRNA transcribed from them are well known. There are 9 such regions known in the bacterial 16S gene and the rRNA transcribed from it (FIGS. 1, 7A). Within each HV region, there are subdomains, described as a-g, for example, within HV3 there are regions HV3a-HV3g (FIGS. 1, 7A). They are called hypervariable because the sequence within each shows substantial diversity among bacterial species. They show such diversity among species because the rRNA gene product is a structural element of the ribosome and the “Hypervariable” regions generally fall into unstructured loop domains and thus show substantial evolutionary drift. Analogously, there are hypervariable regions in both the rDNA and rRNA of the fungal 28S rDNA and rRNA. As for bacteria, among eukaryotes, including fungi, these 28S hypervariable D regions, for example, D1, D2 and D3 (FIG. 7B), reside in unstructured loop regions and thus have developed substantial species-specific sequence diversity.

The sequence-specific diversity resident in the hypervariable regions of 16S and 28S ribosomal genes is well known and is widely used, especially in the context of sequencing, to detect and differentiate microbes in environmental and clinical samples. Such studies of sequence variation among the hypervariable regions have shown that in many cases, a single hypervariable region does not display enough sequence diversity to differentiate a species of bacteria from all others. In those cases, multiple hypervariable regions are sequenced concurrently. The present invention is based on a similar conceptual approach. However, rather than using next generation sequencing to measure sequence diversity among multiple hypervariable regions, a combination of multiplex amplification and highly multiplex sequence analysis by microarray hybridization provides the ability to detect and discriminate complex mixtures in a way that is much simpler, faster and less expensive than a sequencing-based alternative.

Specifically, fluorescently-labeled ribosomal hypervariable region amplicons are hybridized to a plurality of nucleic acid probes (see Tables 3A-3N), each probe in the plurality having a sequence corresponding to a sequence determinant resident in an amplicon and each designed to bind to a specific microbial species, for example, bacterial and fungal species, and each probe attached to a microarray. The microarray is washed at least once and the microarray is imaged to detect at least one fluorescent signal each from the two or more fluorescently-labeled bacterial or fungal amplicons with concurrent analysis of hybridization of two or more hypervariable regions in the same assay, thereby detecting the bacterial and/or fungal species in the sample.

Thus, the amplification with hybridization methods described herein are useful for resolving specific bacterial and fungal species in a sample. Up to four hypervariable regions may be amplified as a single multiplex amplification assay, that is, 16S-HV1, 16S-HV3, 16S-HV6 and 28S-D2. Simultaneous hybridization-based analysis of sequence determinants in multiple Hypervariable regions, is used to discriminate among species in a syndromic panel of microbial contaminants known to be causative for clinical presentation, such as a urinary tract infection or a blood infection or wound infection, where each of the nucleic acid probes is attached at a specific position on a microarray support.

Also provided is hybridizing the fluorescently-labeled amplicons to a sub-set of several nucleic acid probes in parallel, resident on the same microarray. Each of the probes comprising such a sub-set is specific to at least one sequence determinant within one of the ribosomal Hypervariable regions. For example, four hypervariable regions may be amplified, such as 16S-HV1, 16S-HV3, 16S-HV6 for bacteria and 28S-D2 or, alternatively, ITS2 for fungi. There may be, correspondingly, at least four probes provided on the microarray as a set, at least one such probe per amplified region. Each probe in that sub-set is designed to be specific for the same species.

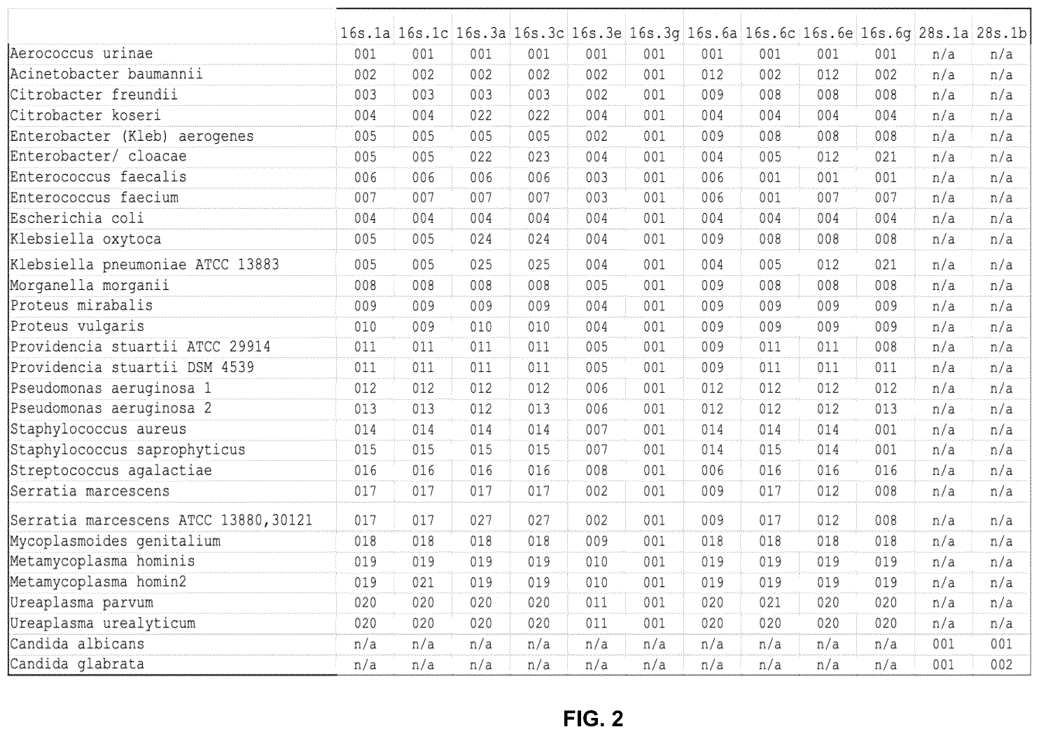

The rational for such concurrent analysis of multiple hypervariable sites is shown in in FIG. 2, for a representative panel of pathogens which are associated with a urinary tract infection (UTI), comprising 24 bacteria and 2 fungal species (left column of FIG. 2). For each species, a unique probe may be designed which binds specifically to one Hypervariable region subdomain. In order to obtain a high level of species-specific hybridization, a set of species-specific hybridization probe is designed for each species. Each member of that set of probes binds to one Hypervariable subdomain sequence presented in that species. Each probe has a unique sequence and is identified by a unique probe Identifier. All such probes are manufactured into a single microarray and made ready to bind to the product of Hypervariable region amplification.

In the representative example of FIG. 2, each species of a UTI pathogen panel is identified by its hybridization to a set of probes which target some or all Hypervariable subdomains. For example, as shown in FIG. 2, experimental hybridization analysis for the species Enterococcus faecium is identified by its binding to 10 unique probes, one each from regions 16s.1a, 16s. 1c, 16s.3a, 16s3c, 16s3e, 16S.3g, 16s.6a, 16s6c, 16s6e, 16S.6g. That pattern of amplified nucleic binding to the panel of 10 unique sequence thus comprises a set (or vector) of concurrent hybridization reactions. Such concurrent binding is the basis for the method of species detection and speciation of the present invention.

The methods provided herein utilize both rDNA and rRNA target analytes. It is well known in the art that individual hypervariable regions (HV) of the ribosomal gene or its RNA product contain sequences which detect the presence of a species, i.e. have good inclusivity, but in many instances cannot unambiguously resolve the species of interest from other contaminating microbes or host factors, i.e. have poor exclusivity. In particular, many bacterial or fungal sequences presenting in a single HV region might display 90% inclusivity, that is, fail to detect the species of interest 10% of the time, but only deliver 50% exclusivity, that is, inadvertently misidentify as a false positive, a contaminating species as the species of interest 50% of the time.

The several hypervariable regions of the rRNA gene are known to have evolved semi-independently and thus are only weakly correlated. Thus, if (m) such weakly correlated HV sites are analyzed in parallel, as provided herein, if the statistical likelihood of a false positive were 0.5 for each, the likelihood of a false positive among (m) such sites measured as multiplex hybridization could approach 0.5^{m}. For instance, for m=3 quasi-independent HV sites, the false positive rate would thus decrease from 50% to 12%, while the inclusivity would increase from 90% (1-10⁻¹) to >99% (1-10⁻³). Such multiplex amplification of the multiple hypervariable regions presented in rRNA or rDNA, followed by hybridization of the resulting amplicons to a species-specific probe sub-set generates a substantial improvement of inclusivity and exclusivity and is central to the present invention.

The methods provided herein are useful for testing a biological specimen for the presence of bacteria and/or fungi. A sample is obtained from a human subject or other mammal, or from matter such as a swab in previous contact with the subject from which total nucleic acids are isolated. At least one amplification reaction is performed on the total nucleic acid using at least one fluorescently-labeled primer pair. Preferably, two or more fluorescently-labeled primer pairs, which are selective for two or more ribosomal hypervariable regions, are used to generate two or more fluorescently-labeled hypervariable rDNA or rRNA amplicons as described herein.

The fluorescently-labeled rDNA or rRNA amplicons are hybridized to nucleic acid probes each of which has a sequence complementary to a sequence determinant in at least one hypervariable region among the syndromic panel of microbes relevant to a specific clinical indication, such as urinary tract infection or blood infection or wound infection. The specific pattern of the fluorescent signal on the microarray support, among a panel of species-specific probes, differentiates the bacteria and/or fungal species present in the human or animal specimen and identifies the specific clinical indication.

An internal control may be incorporated into the methods provided herein. Particularly, one or more additional genes may be amplified and hybridized with the bacterial and/or fungal ribosomal DNA (rDNA) or ribosomal RNA (rRNA). For example, but not limited to, the human RNAseP gene (Table 3M) may be used as an internal control. Alternatively, the methods provided herein are useful for obtaining information about these additional genes within the bacterial and/or fungal species in the sample, for example, but not limited to, which, if any, of the genes or their RNA products confer antibiotic resistance to the identified bacteria and/or fungi.

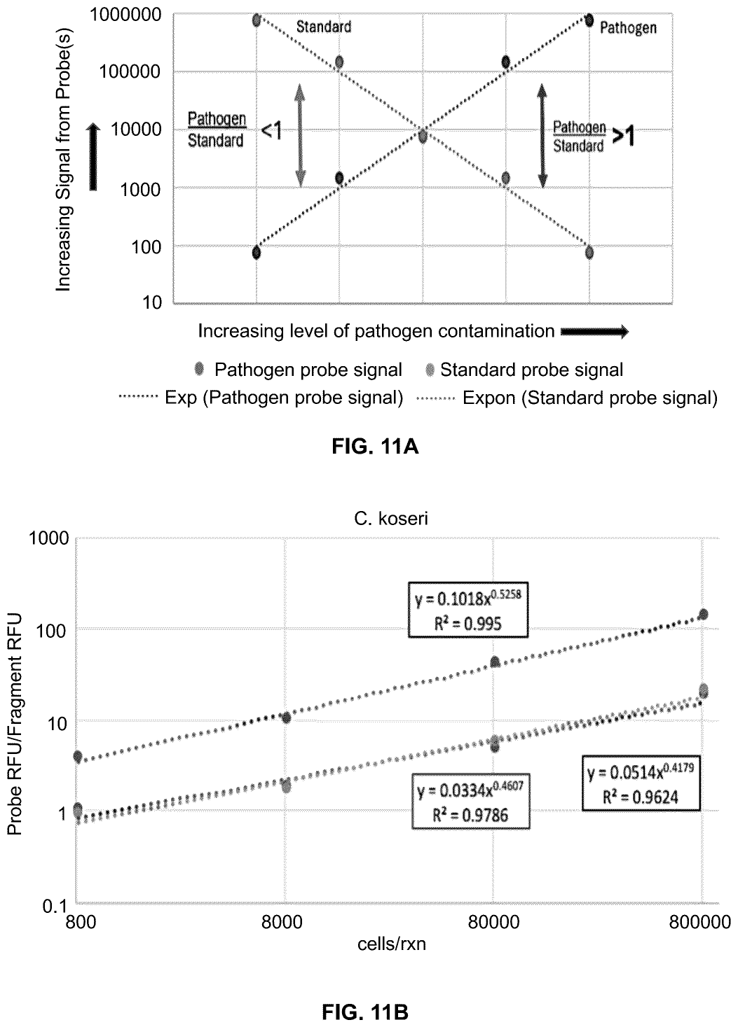

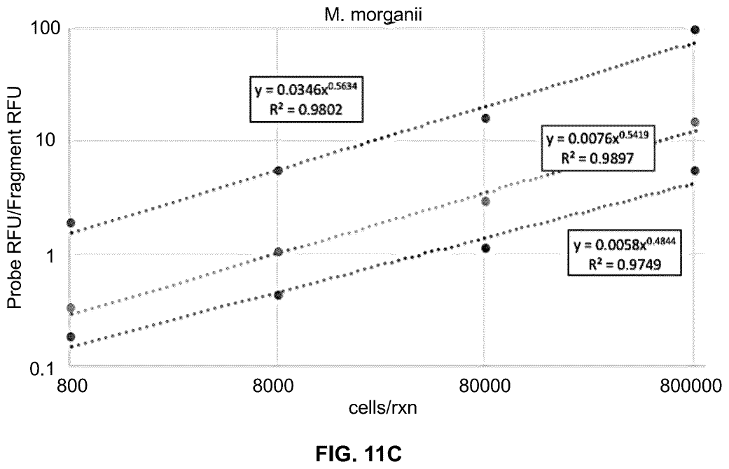

Additionally, a quantitative reference standard (Table 3N) may be incorporated into the methods provided herein to quantify the abundance of each species in the sample. A known number of copies of the quantitative reference standard is added to the amplification step and is similar in sequence to the pathogen DNA regions being amplified, for example, but not limited to, the probe sequences for 16S-HV3-6 (Tables 3C-3J). The abundance of each species detected in the multiplex amplification assay is obtained from the measured ratio of the hybridization signal from a species specific probe to that of a corresponding probe which binds only to the Quantitative Reference Standard. A hybridization signal ratio [Pathogen/Standard RFU] is obtained from those data and may be uniquely linked to the ratio of pathogen gene copies present in a sample relative to the (known) number of standard copies per reaction (FIGS. 11A-11C). The method is applicable to any similar bacterial species or to any similar fungal species or other eukaryotic species of concern.

The following examples are given for the purpose of illustrating various embodiments of the invention and are not meant to limit the present invention in any fashion.

### Example 1

### Multiplex PCR Probe Design and Amplification of Hypervariable Region Targets

PCR is performed on the rDNA Hypervariable regions of purified bacteria and fungi at the cycling conditions shown in Table 1. The PCR is performed as an asymmetric endpoint PCR reaction. Primer pairs for each of the N=4 elements of the multiplex are shown in Table 2.

TABLE 1
PCR Cycling Conditions for Multiplex Amplification of
HV1, HV3, HV6, 23S HV

| PCR | | | |
|---|---|---|---|
| Steps | Temp. | Time | Cycles |
| 1 | 95° C. | 4 minutes | 1 |
| 2 | 95° C. | 30 seconds | 45 |
| 3 | 55° C. | 30 seconds | |
| 4 | 72° C. | 1 minute | |
| 5 | 72° C. | 7 minutes | 1 |
| 6 | 15° C. | ∞ | 1 |

The multiplex PCR is performed using one fluorescently labeled primer pair for each HV region (16S-HV1, -HV3, -HV6, 28S-D2). Table 2 lists non-limiting examples of primer pairs suitable for the asymmetric endpoint reaction for the entire species set of FIG. 2. Additionally, an additional PCR reaction for the human gene RNAseP or an analogous human gene may be added to the same HV region multiplex as a control. The primers each may have a 5′-terminal fluorescent label, for example, but not limited to, the fluorophores CY3 or CY5. When needed, the same primers used for PCR, with the addition of reverse transcriptase to the PCR reaction, may be used to amplify rRNA. A related multiplex reaction may also be driven by isothermal amplification of the cellular rRNA, via a version of Transcription Mediated Amplification, sometimes referred to as NASBA. Those NASBA primers are based on the primer sequences used for PCR.

TABLE 2
PCR Primers 16S: HV1,HV3, HV6/28S: D2/RNaseP

SEQ
ID

| | |
|---|---|
| Primer Sequences | NO: |

16S rDNA Hypervariable region 1 forward and reverse primers

| | | |
|---|---|---|
| 16S HV1 FP-1.1 | TTTAGAGTTTGATCMTGGCTCAG | 1 |
| 16S HV1 RP C3-1.1a | /5Cy3/TTTACATTACTCACCCGTCCGC | 2 |
| 16S HV1 RP C3 1.1b | /5Cy3/TTTGTGTTACTCACCCGTYCGC | 3 |

16S rDNA Hypervariable region 3 forward and reverse primers

| | | |
|---|---|---|
| 16S HV3 FP 1.1 | TTTCACATTGGRACTGAGACACGG | 4 |
| 16S HV3 RP C3 1.1 | /5Cy3/TTTTGTATTACCGCGGCTGCTGGCA | 5 |

16S rDNA Hypervariable region 6 forward and reverse primers

| | | |
|---|---|---|
| 16S HV6 FP 1.1 | TTCGCACAAGCGGTGGAGCATGT | 6 |
| 16S HV6 RP C3 2.1 | /5Cy3/TTTACGAGCTGACGACAGCCATGCA | 7 |

28S rRNA forward and reverse primer

| | | |
|---|---|---|
| 28S FP 1.1 | TTGTACGTGAAATTGTTGAAAG | 8 |
| 28S RP C3 1.1 | /5Cy3/TTAAACCGCAGTCCTCRGTC | 9 |

RNaseP forward and reverse primer

| | | |
|---|---|---|
| RNaseP FP 1.1 | TTTGTTTGCAGATTTGGACCTGCGAGCG | 10 |
| RNaseP RP C3 1.1 | 5Cy3/TTTAAGGTGAGCGGCTGTCTCCACAAGT | 11 |

### Example 2

### Microarray Design to Support Hybridization Analysis of Bacterial and Fungal Amplicons

Bacterial Hypervariable region amplicons, as in Example 1, are hybridized to a microarray or a microarray support, such as, but not limited to, a microarray with a functionalized solid surface, to which a plurality of bacterial and fungal nucleic acid probes are directly or indirectly covalently attached. The attachment site correlates to a specific nucleic acid sequence. The nucleic acid probes may be indirectly covalently attached via linker, for example, a bifunctional oligonucleotide linker, such as, but not limited to, the oligothymidine linker OLIGO-T, which is covalently attached at one terminal nucleotide to the functionalized and covalently crosslinked to at least one nucleic acid probe at the other terminus.

### Methods of Primer and Probe Design

The approach is as follows. Define a specific set (N) of bacteria or fungi to be analyzed, typically 10 to 100 species such as the syndromic panel suitable for urinary tract infection testing in FIG. 1. The hypervariable (HV) regions (HV1, HV3, HV6) of the bacterial 16S gene and (D2) of the Fungal 28S gene can then be amplified by a single set of four universal primers as in Table 2 for PCR or RT-PCR with the exception that the 28S D2 reverse primer is semi-specific for Candida spp.

Generally, each HV region is amplified by a single primer pair, found to be common to the “Universal” ribosomal gene sequence flanking each HV region. Specific examples are shown in Table 2, that is, one “Universal” primer pair each for the several HV regions to be used in the assay. At each of the HV regions, a gene sequence within each HV region is picked which is unique or nearly unique to each species in the set. Such “highly discriminating” sequences are used to design hybridization probes which are used to detect the presence of the unique sequence for that bacterium of fungi among the set of (N) species. Representative examples of such “highly discriminating” probes are shown in Tables 3A-3M. Updated probe sequence content is presented in a bold italic.

It is generally possible to find a “highly discriminating” sequence for a species in each 16S Hypervariable region (HV1, HV3, HV6) and fungal 28S D2. As a result, discrimination of a bacterial species is not based on hybridization to a single probe, but is instead based on analysis of hybridization to a probe sub-set, which for any species of interest comprises at least one “highly discriminating” probe in HV1 and HV3 and HV6 for bacterial species and at least one “highly discriminating” probe in D2 for fungal species, where species discrimination is obtained by concurrent analysis of hybridization to probes from all three HV regions.

Having found amplification primers and associated highly discriminating probe sequences in each of the 4 amplicons and having designed hybridization probes for them in all (N) species in the set, a multiplex assay is then performed comprising three steps. Firstly, multiplex PCR or RT-PCR or isothermal amplification of all Hypervariable regions under study. Secondly, hybridization of the resulting set of amplified nucleic acid products to a microarray. Thirdly, detect and discriminate all members of the set of N species, based on simultaneous sequence analysis by hybridization to the amplicon products from Hypervariable regions in parallel.

Tables 3A-3B list non-limiting examples of nucleic acid probes selective for sequence determinants complementary to specific bacterial species, as detected per the present invention, in the 16S hypervariable regions 1a and 1c. Tables 3C-3F list non-limiting examples of nucleic acid probes selective for sequence determinants complementary to specific bacterial species as detected per the present invention, in 16S hypervariable regions 3a, 3c, 3e, and 3g. Tables 3G-3J list non-limiting examples of nucleic acid probes selective for sequence determinants complementary to specific bacterial species as detected per the present invention in 16S hypervariable regions 6a, 6c, 6e, and 6g. Tables 3K-3L lists non-limiting examples of nucleic acid probes selective for sequence determinants complementary to specific fungal species as detected per the present invention in the fungal 28S-D2 hypervariable regions 1a and 1b. Table 3M lists non-limiting examples of nucleic acid probes selective for sequence determinants complementary to human RNAseP gene controls. Table 3M is an example of a quantitation reference standard for the 16S gene.

TABLE 3A
16S rDNA Hypervariable region 1a probe sequences (Bacterial)

| Probe | Sequence | SEQ ID NO: |
|---|---|---|
| 16s.HV1a.001 | TTTTTTTGAACCGACGAAGTGCTCTTTTTT | 12 |
| 16s.HV1a.002 | TTTTTATTGGGAAGGTAGCTTGTATTTCTT | 13 |
| 16s.HV1a.003 | TTTTTTTATGCACAGAGGAGCTATTTATTT | 14 |
| 16s.HV1a.004 | TTTTTCTACAGGAAGCAGCTTGCCTTTTTT | 15 |
| 16s.HV1a.005 | TTTTTTTCAGCACAGAGAGCTTATTTTTTT | 16 |
| 16s.HV1a.006 | TTTTTGCTTCTTTCCTCCCGAGTGTCTTTT | 17 |
| 16s.HV1a.007 | TTTTCGCTTCTTTTTCCACCGGAGTCTTTT | 18 |
| 16s.HV1a.008 | TTTTTATTAACAGGGAGAAGCTATTTTTTT | 19 |
| 16s.HV1a.009 | TTTTTAGTAACAGRAGAAAGCTCTATTTTT | 20 |
| 16s.HV1a.010 | TTTATTTGGTAACAGAAGAAAGCATTTTTT | 21 |
| 16s.HV1a.011 | TTTTTTTTTAACAGGGGAAGCTTATTATTT | 22 |
| 16s.HV1a.012 | TTTTTACTGGATGAAGGGAGCTATTTTTTT | 23 |
| 16s.HV1a.013 | TTTTTATCTTATGAAGGGAGCTATTTTTTT | 24 |
| 16s.HV1a.014 | TTTTTTTTACGGACGAGAAGCCCTTTTTTT | 25 |
| 16s.HV1a.015 | TTTTTTTGAACAGATAAGGAGCTATTTTTT | 26 |
| 16s.HV1a.016 | TTTTTTTGGTTTGGTGTTTACATCTTTTTT | 27 |
| 16s.HV1a.017 | TTTTTTTCTGCACAGGGGAGCTATTTTTTT | 28 |
| 16s.HV1a.018 | TTTTTTTTATCGGAAGTAGCAATTTTTTTT | 29 |
| 16s.HV1a.019 | TTTTTTTGAGCGAGGTTAGCAATTTTTTTT | 30 |
| 16s.HV1a.020 | TTTTTCTCGAACGAAGCCTTTTAGTTTTTT | 31 |

TABLE 3B
16S rDNA Hypervariable region 1c probe sequences (Bacterial)

| Probe | Sequence | SEQ ID NO: |
|---|---|---|
| 16s.HV1c.001 | TTTTTTTTTGCACTTCTGACGTATTTTTTT | 32 |
| 16s.HV1c.002 | TTTTTTTCTGCTACCGGACCTATTTTTTTT | 33 |
| 16s.HV1c.003 | TTTTTTTTCTTGCTCCTTGGGTTTTTTTTT | 34 |
| 16s.HV1c.004 | TTTTTTTCTTGCTGCTTTGCTGATTTTTTT | 35 |
| 16s.HV1c.005 | TTTTTTTTCTTGCTCTCGGGTTTTTTTTTT | 36 |
| 16s.HV1c.006 | TTTTGCTTGCACTCAATTGGAAAGTCTTTT | 37 |
| 16s.HV1c.007 | TTCTTTGCTTGCTCCACCGGAAAACTTTTT | 38 |
| 16s.HV1c.008 | TTTTTTCTCTTCTCTGCTGACGTTTTTTTT | 39 |
| 16s.HV1c.009 | TTTTTTTCTGCTTTCTTGCTGACTTTTTTT | 40 |
| 16s.HV1c.011 | TTTTTTTTTTGCTTCTCGCTGATTTTTTTT | 41 |
| 16s.HV1c.012 | TTTTTTTTTGCTCCTGGATTCATTTTTTTT | 42 |
| 16s.HV1c.013 | TTTTTTTTCTTGCCTTGGATTCATTTTTTT | 43 |
| 16s.HV1c.014 | TTTTTTCTGCTTCTCTGATGTTATTTTTTT | 44 |
| 16s.HV1c.015 | TTTTTTTCTGCTCCTTTGACGTTTTTTTTT | 45 |
| 16s.HV1c.016 | TTTTTTCGTTTACACTAGACTGTCCTTTTT | 46 |
| 16s.HV1c.017 | TTTTTTTTCTTGCTCCCTGGGTTTTTTTTT | 47 |
| 16s.HV1c.018 | TTTTTTTGTAGCAATACTTTAGATTTTTTT | 48 |
| 16s.HV1c.019 | TTTTTTCCAGCAATAACCTAGCGGTTTTTT | 49 |
| 16s.HV1c.020 | TTTTTTTGCTTAGTGGTGAACGCTTTTTTT | 50 |

TABLE 3C
16S rDNA Hypervariable region 3a probe sequences (Bacterial)

| Probe | Sequence | SEQ ID NO: |
|---|---|---|
| 16s.HV3a.001 | TTTTTTTACAAATTGGAGAGTAATCTTTTT | 51 |
| 16s.HV3a.002 | TTTTTTTGCTACTTTAGTTAATACTCTTTTTT | 52 |
| 16s.HV3a.003 | TTTTTTACAGGAAGGCGTTGTGTATTTTTT | 53 |
| 16s.HV3a.004 | TTTTTTCAGGGAGTAAAGTTAATATTTTTT | 54 |
| 16s.HV3a.005 | TTTTTTTGAAGGCGTTAAGGTTATCTTTTT | 55 |
| 16s.HV3a.006 | TTTTTTGAACAAGGACGTTAGTAATTTTTT | 56 |
| 16s.HV3a.007 | TTTTTTTCAAGGATGAGAGTAACCTTTTTT | 57 |
| 16s.HV3a.008 | TTTTTTTTAAGGTGTCAAGGTTAAATTTTTTT | 58 |
| 16s.HV3a.009 | TTTTTTGTGATAAGGTTAATACCCATTTTT | 59 |
| 16s.HV3a.010 | TTTTTGTGATAAAGTTAATACCTTCTTTTT | 60 |
| 16s.HV3a.011 | TTTTTTTAAGGCGTTGATGTTAAATTTTTT | 61 |
| 16s.HV3a.012 | TTTTTTTCAAGGGCAGTAAGTTAAATTTTTTT | 62 |
| 16s.HV3a.014 | TTTTTTGAAGAACATATGTGTAAGCTTTTT | 63 |
| 16s.HV3a.015 | TTTTCTAAGAACAAAYGTGTAAGCTTTTTT | 64 |
| 16s.HV3a.016 | TTTTTTTTAGAACGTTGGTAGGATTTTTTTTT | 65 |
| 16s.HV3a.017 | TTTTTTTAGGTGGTGARCTTAATTTTTTTT | 66 |
| 16s.HV3a.018 | TTTTTCAAGAATGACTCTAGCAGTTTTTTT | 67 |
| 16s.HV3a.019 | TTTTTTCAAGAACATTTGCAATAGTCTTTTTT | 68 |
| 16s.HV3a.020 | TTTTTTTCAGAAACGCTAAGATAGTTCTTTTT | 69 |
| 16s.HV3a.022 | TTTTTCTTGGAAGGTGTTGTGTATTTCTTT | 70 |
| 16s.HV3a.024 | TTTTTTCTAAGGGAGTGAGGTTAAATCTTTTT | 71 |
| 16s.HV3a.025 | TTTTTTTTGAAGGCGATGAGGTTTTTTTTT | 72 |
| 16s.HV3a.026 | TTTTTTTAAGGCGATAAGGTTAATTTTTTT | 73 |
| 16s.HV3a.030 | TTTTTTCTAAGGCGGTGAGGTTTTTTTTTT | 74 |
| 16s.HV3a.101 | TTTTTCTATGTGAGTTAAGTTACTTTTTTT | 75 |

TABLE 3D
16S rDNA Hypervariable region 3c probe sequences (Bacterial)

| Probe | Sequence | SEQ ID NO: |
|---|---|---|
| 16s.HV3c.001 | TTTTTTTCAACTGCTCCAGTCTTTTTTTTT | 76 |
| 16s.HV3c.002 | TTTTTTCTATACCTAGAGATAGTGTTTTTTTT | 77 |
| 16s.HV3c.003 | TTTTTTTTGCAGCGATTGACGTATTTTTTT | 78 |
| 16s.HV3c.004 | TTTTTTTTTACCTTTGCTCATTGATTTTTTTT | 79 |
| 16s.HV3c.005 | TTTTTTTTTAACCTTGGCGATTGAATTTTTTT | 80 |
| 16s.HV3c.006 | TTTTTTTAACTGAACGTCCCCTGATTTTTT | 81 |
| 16s.HV3c.007 | TTTTTTTTAACTGTTCATCCCTTGTTTTTT | 82 |
| 16s.HV3c.008 | TTTTTTTTAACCTTGGCAATTGACTTTTTT | 83 |
| 16s.HV3c.009 | TTTTTTGGTTAATACCCTTRTCATCTTTTT | 84 |
| 16s.HV3c.010 | TTTTCTAGTTAATACCTTTGTCATCTTTTT | 85 |
| 16s.HV3c.011 | TTTTTTTTACCATCAACGATTGATTTTTTT | 86 |
| 16s.HV3c.012 | TTTTTCTATACCTTGCTGTTTTGATTTTTT | 87 |
| 16s.HV3c.013 | TTTTTTCTATACCTTGCTGTTTGATTTTTTTT | 88 |
| 16s.HV3c.014 | TTTTTTTTGTGCACATCTTGACTTTTTTTT | 89 |
| 16s.HV3c.015 | TTTTTTTAACTGTGTACGTCTTGTTTTTTT | 90 |
| 16s.HV3c.016 | TTTCTTCGGAAAATCTACCAAGTGTTTTTT | 91 |
| 16s.HV3c.017 | TTTTTCAATACGYTCATCAATTGAATTTTT | 92 |
| 16s.HV3c.018 | TTTTTTAGGCTGGAGTTTGACTTTTTTTTT | 93 |
| 16s.HV3c.019 | TTTTTTTCGATTGCAGACTGACGTTTTTTTTT | 94 |
| 16s.HV3c.020 | TTTTTTTAATGATTTTAGTTTGACTTTTTTTT | 95 |
| 16s.HV3c.023 | TTTTTTTTCGCAGCAATTGACGCTTTTTTT | 96 |
| 16s.HV3c.024 | TTTTTTTTAACCTTATTCATTGACTCTTTTTT | 97 |
| 16s.HV3c.025 | TTTTTCTATAACCTCATCGATTGTTTTTTT | 98 |
| 16s.HV3c.026 | TTTTTTTTAACCTTGTCGATTGAATTTTTT | 99 |
| 16s.HV3c.101 | TTTTTTCACTACCTTAGCTCATATTTTTTT | 100 |

TABLE 3E
16S rDNA Hypervariable region 3e probe sequences (Bacterial)

| Probe | Sequence | SEQ ID NO: |
|---|---|---|
| 16s.HV3e.001 | TTCTTTAGGTATCTTACCAGAAACTTTTTT | 101 |
| 16s.HV3e.002 | TTTTTTCTACGTTACTCGCAGAATTTTTTT | 102 |
| 16s.HV3e.003 | TTTTTTTTCGGTATCTAACCAGACTTTTTT | 103 |
| 16s.HV3e.004 | TTTTTTCTACGTTACCCGCAGAATTTTTTT | 104 |
| 16s.HV3e.005 | TTTTTTTTGTTACCGACAGAAGATTTTTTT | 105 |
| 16s.HV3e.006 | TTTTTTTCGTTACCAACAGAATACTTTTTT | 106 |
| 16s.HV3e.007 | TTTTTTCTACGGTACCTAATCAGTTTTTTT | 107 |
| 16s.HV3e.008 | TTTTTTCTACGGTAACTAACCAGTTTTTTT | 108 |
| 16s.HV3e.009 | TTTTTTTACTTTGAATAAGTGACGTTTTTT | 109 |
| 16s.HV3e.010 | TTTTTTTGGTACCTTGTCAGAAATTTTTTT | 110 |
| 16s.HV3e.011 | TTTTCTACTGTACCATTTGAATAATCTTTT | 111 |

TABLE 3F
16S rDNA Hypervariable region 3g
probe sequence (Bacterial)

| Probe | Sequence | SEQ ID |
|---|---|---|
| | | NO: |
| 16s.HV3g.001 | TTTTTTTACCTACGG | 112 |
| | GAGGCAGCTTTTTTT | |
| 16s.HV3g.101 | TTTTTTCTCCTGCAG | 113 |
| | GAGACGGTCTTTTTT | |

TABLE 3G
16S rDNA Hypervariable region 6a
probe sequences (Bacterial)

| Probe | Sequence | SEQ ID NO: |
|---|---|---|
| 16s.HV6a.001 | TTTTTTTCTTACCAAGTCTTGACTCTTTTT | 114 |
| 16s.HV6a.004 | TTTTTTTCACCTGGTCTTGACACTTTTTTT | 115 |
| 16s.HV6a.006 | TTTTTCTCCTTACCAGGTCTTGATTTTTTT | 116 |
| 16s.HV6a.009 | TTTTTTTCCTTACCTACTCTTGTTTTTTTT | 117 |
| 16s.HV6a.012 | TTTTTTTCTACCTGGCCTTGACTCTTTTTT | 118 |
| 16s.HV6a.014 | TTTTTTTTTACCAAATCTTGACACTTTTTT | 119 |
| 16s.HV6a.018 | TTTTTTTCTTACCTAGACTTGACTCTTTTT | 120 |
| 16s.HV6a.019 | TTTTTTTACCTTACCCACTCTTGTTTTTTT | 121 |
| 16s.HV6a.020 | TTTTTTCCTTACTTAGGTTTGACACTTTTT | 122 |

TABLE 3H
16S rDNA Hypervariable region 6c probe sequences (Bacterial)

| Probe | Sequence | SEQ ID NO: |
|---|---|---|
| 16s.HV6c.001 | TTTTTCTCTTTGACCACTCTAGATTTTTTT | 123 |
| 16s.HV6c.002 | TTTTTTTATACTAGAAACTTTCCATTTTTT | 124 |
| 16s.HV6c.004 | TTTTTTTCCCACGGAAGTTTTCATTTTTTT | 125 |
| 16s.HV6c.005 | TTTTTTTCCCACAGAACTTTCCTTTTTTTT | 126 |
| 16s.HV6c.008 | TTTTTTTCCCAGAGAACTTAGCTTTTTTTT | 127 |
| 16s.HV6c.009 | TTTTTTCTCAGCGAATCCTTTAGTTTTTTT | 128 |
| 16s.HV6c.011 | TTTTTTATCAGAGAATTTAGCAGACTTTTT | 129 |
| 16s.HV6c.012 | TTTTTTTTGCTGAGAACTTTCCTTTTTTTT | 130 |
| 16s.HV6c.014 | TTTTTTAATCCTTTGACAACTCTATTTTTT | 131 |
| 16s.HV6c.015 | TTCTTAATCCTTTGAAAACTCTAGTTCTTT | 132 |
| 16s.HV6c.016 | TTTTTTTTTCCTTCTGACCGGCTCTTTTTT | 133 |
| 16s.HV6c.017 | TTTTTTATTCCAGAGAACTTTCCTTTTTTT | 134 |
| 16s.HV6c.018 | TTTTTTTTCCTTGGCAAAGTTATGTTTTTT | 135 |
| 16s.HV6c.019 | TTTTTTTTCCTTCGCAAAGCTATATTTTTT | 136 |
| 16s.HV6c.020 | TTTTTTTCATTGCGACGCTATAGTTTTTTT | 137 |
| 16s.HV6c.021 | TTTTTTCTTTGCGATGCTATAGTTTTTTTT | 138 |
| 16s.HV6c.026 | TTTTTTTTTCCACAGAACTTAGCTTTTTTT | 139 |
| 16s.HV6c.101 | TTTTTTTCCCTCGGACGTTTACTTTTTTTT | 140 |

TABLE 3I
16S rDNA Hypervariable region 6e probe sequences (Bacterial)

| Probe | Sequence | SEQ ID NO: |
|---|---|---|
| 16s.HV6e.001 | TTTTTTCTGAGCTTTCCCTTCGTTTTTTTT | 141 |
| 16s.HV6e.004 | TTTTTTTTGATGAGAATGTGCCACTTTTTT | 142 |
| 16s.HV6e.007 | TTTTTTTCAGAGCTTCCCCTTCGTTTTTTT | 143 |
| 16s.HV6e.008 | TTTTTTTTATGCTTTGGTGCCCCTTTTTTT | 144 |
| 16s.HV6e.009 | TTTTTTTCATAGAGGAGTGCCTATCTTTTT | 145 |
| 16s.HV6e.011 | TTTTTTTATGCTTTAGTGCCTTTTTTTTTT | 146 |
| 16s.HV6e.012 | TTTTTTTTTGATGGATTGGTGCTCTTTTTT | 147 |
| 16s.HV6e.014 | TTTTCTTATAGAGCCTTCCCCTTCTTTTTT | 148 |
| 16s.HV6e.016 | TTTTTTTTAGGCTTTCTCTTCGTTTTTTTT | 149 |
| 16s.HV6e.018 | TTTTTTCAAACATAATGGAGGTTCTTTTTT | 150 |
| 16s.HV6e.019 | TTTTTTTAGATATAGTGGAGGTTCTTTTTT | 151 |
| 16s.HV6e.020 | TTTTTAAATATAGTTGAGGTTAATATTTTT | 152 |

TABLE 3J
16S rDNA Hypervariable region 6g probe sequences (Bacterial)

| Probe | Sequence | SEQ ID NO: |
|---|---|---|
| 16s.HV6g.001 | TTTTTTTGGACAAAGTGACAGGCTATTTTT | 153 |
| 16s.HV6g.002 | TTTTTTTGGAATCTAGATACAGGCTTTTTT | 154 |
| 16s.HV6g.004 | TTTTTATTGGAACCGTGAGACTTTTTTTTT | 155 |
| 16s.HV6g.007 | TTTTTTTGGCAAAGTGACAGGCTATTTTTT | 156 |
| 16s.HV6g.008 | TTTTTTTTGGAACTCTGAGACATTTTTTTT | 157 |
| 16s.HV6g.009 | TTTTTTATTGGGAACGCTGAGATTTTTTTT | 158 |
| 16s.HV6g.011 | TTTTTTTTTTCGGGAGCTCTGATTTTTTTT | 159 |
| 16s.HV6g.012 | TTTTTTTGAACAGAGACACAGGTTTTTTTT | 160 |
| 16s.HV6g.013 | TTTTTTTGAACTCAGACACAGGCTTTTTTT | 161 |
| 16s.HV6g.016 | TTTTTTTAGCAGAAGTGACAGTCTTTTTTT | 162 |
| 16s.HV6g.018 | TTTTTTTTAGGTTAACCGAGTGTTTTTTTT | 163 |
| 16s.HV6g.019 | TTTTTTTTAGGTTATCGGAGTGTTATTTTT | 164 |
| 16s.HV6g.020 | TTTTTTTGGTTAACAATATGACATTTTTTT | 165 |
| 16s.HV6g.021 | TTTTTATTGGAACTGTGAGACATTTTTTTT | 166 |

TABLE 3K
28S D2 rDNA probe region 1a sequences (Fungal)

| Probe | Sequence | SEQ ID NO: |
|---|---|---|
| 28s.1a.001 | TTTTTTCGGCCAGCAT | 167 |
| | CAGTTTGGTCTTTTTT | |

TABLE 3L
28S D2 rDNA probe region 1b sequences (Fungal)

| Probe | Sequence | SEQ ID NO: |
|---|---|---|
| 28s.1b.001 | TTTTTTTTCACGGCTT | 168 |
| | CTGCTGTGCTTTTTTT | |
| 28s.1b.002 | TTTTTTTTCTCTGCGC | 169 |
| | CTCGGTGTTTTTTTTT | |

TABLE 3M
Negative control, Positive RNaseP control and
RNaseP control fragment probe

| | | SEQ ID |
|---|---|---|
| Probe | Sequence | NO: |
| neg.cont | TTTTTCTACTACCTAT | 170 |
| | GCTGATTCACTCTTTT | |
| RNaseP.cont | TTTTTCTGACCTGAAG | 171 |
| | GCTCTGCGCTTTTT | |
| RNaseP.spk. | TTTTTCTGTCCAGATG | 172 |
| cont | GCACAGCGCTTTTT | |

TABLE 3N
16S Quantitation reference standard-790 bp

| | | SEQ |
|---|---|---|
| | | ID |
| Probe | Sequence | NO: |
| 16S- | TCTGAGAGGATGACCAGCCACACTGGAACTGA | 173 |
| HV3-6. | TGGGGAATATTGCACAATGGGCGCAAGCCTGA | |
| Quant | TGCAGCCATGCCGCGTGTATGAAGAATGCCTT | |
| ref | CGGGTAGTAAAGTACTTTCAGTGGGGAGGATG | |
| | TGAGTTAAGTTACTACCTTAGCTCATAGACCTT | |
| | ACCAGCAGTAGAAGCACCGGCTAACTCCGTGC | |
| | CAGCAGCCGCGGTAATACGGAGGGTGCAAGC | |
| | GTTAATCGGAATTACTGGGCGTAAAGCGCACG | |
| | CAGGCGGTTTGTTAAGTCAGATGTGAAATCCC | |
| | CGGGCTCAACCTGGGAACTGCATCTGATACTG | |
| | GCAAGCTTGAGTCTCGTAGAGGGGGGTAGAAT | |
| | TCCAGGTGTAGCGGTGAAATGCGTAGAGATCT | |
| | GGAGGAATACCGGTGGCGAAGGGGCCCCCT | |
| | GGACGAAGACTGACGCTCAGGTGCGAAAGCG | |
| | TGGGGAGCAAACAGGATTAGATACCCTGGTAG | |
| | TCCACGCCGTAAACGATGTCGACTTGGAGGTT | |
| | GTGCCCTTGAGGCGTGGCTTCCGGAGCTAAC | |
| | GCGTTAAGTCGACCGCCTGGGGAGTACGGCC | |
| | GCAAGGTTAAAACTCAAATGAATTGACGGGGG | |
| | CCCGCACAAGCGGTGGAGCATGTGGTTTAATT | |
| | CGATGCAACGCGAAGAACCTAACCTGCTCTAG | |
| | ACATCCTCGGACGTTTACAGAGCTGAGACTGT | |
| | GACTTCGAGAACAGTGAGTCAGGTGCTGCATG | |
| | GCTGTCGTCAGCTCGTGTTGTGAAAT | |

### Example 3

### Detection and Discrimination Among Microbial Species Known to be Causative for UTI

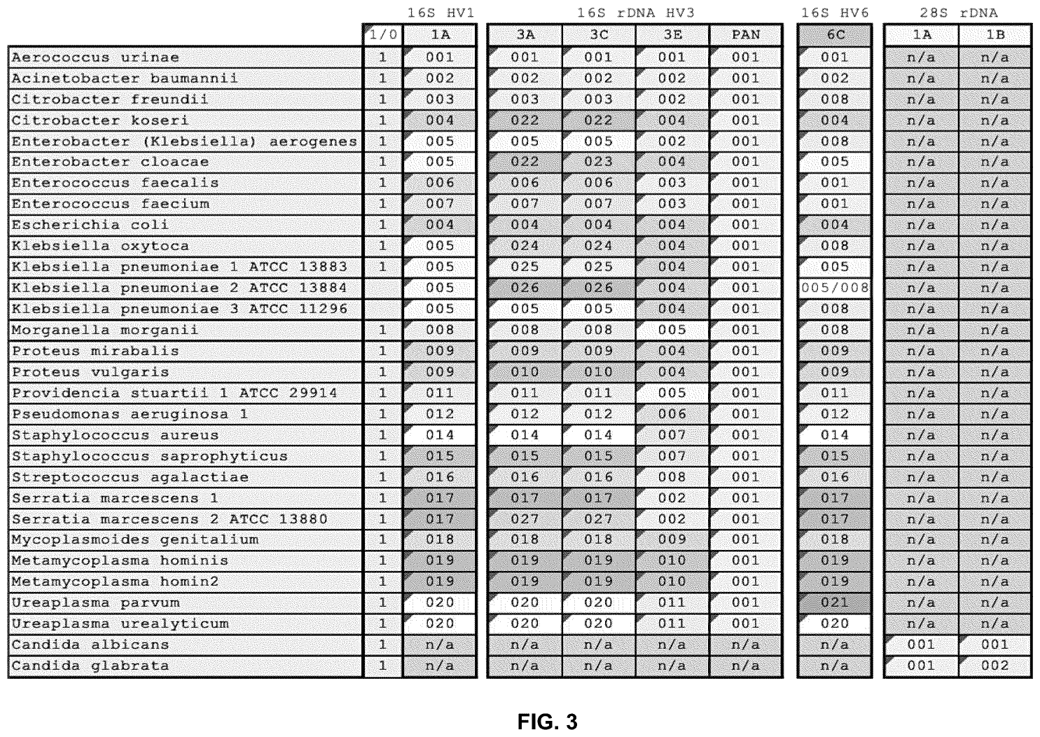

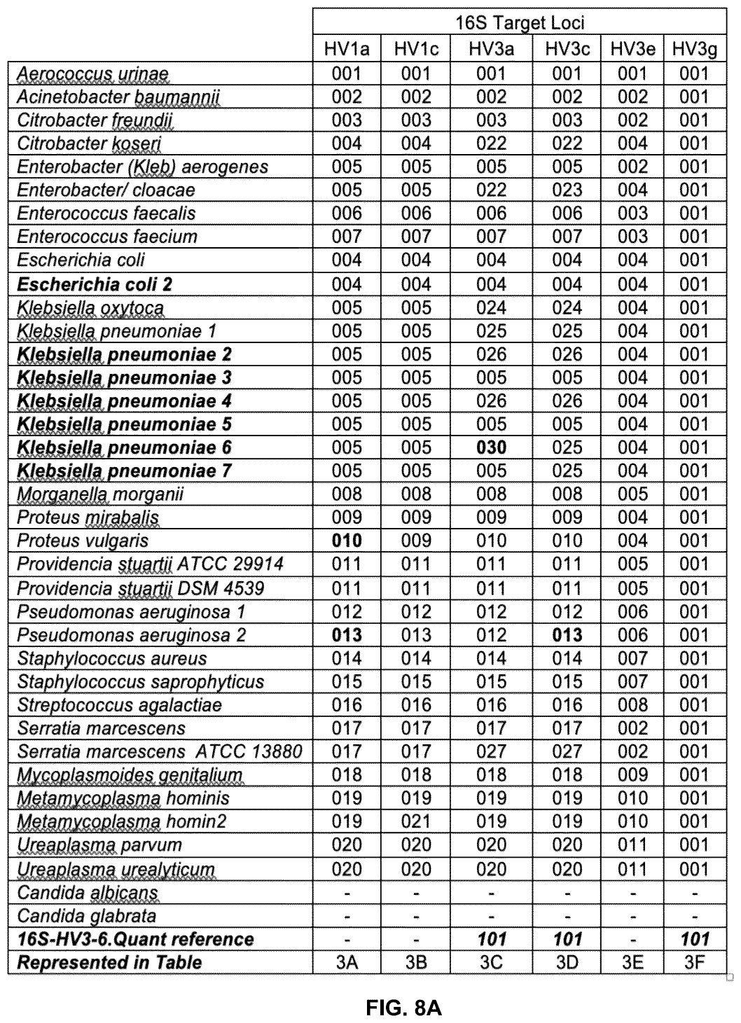

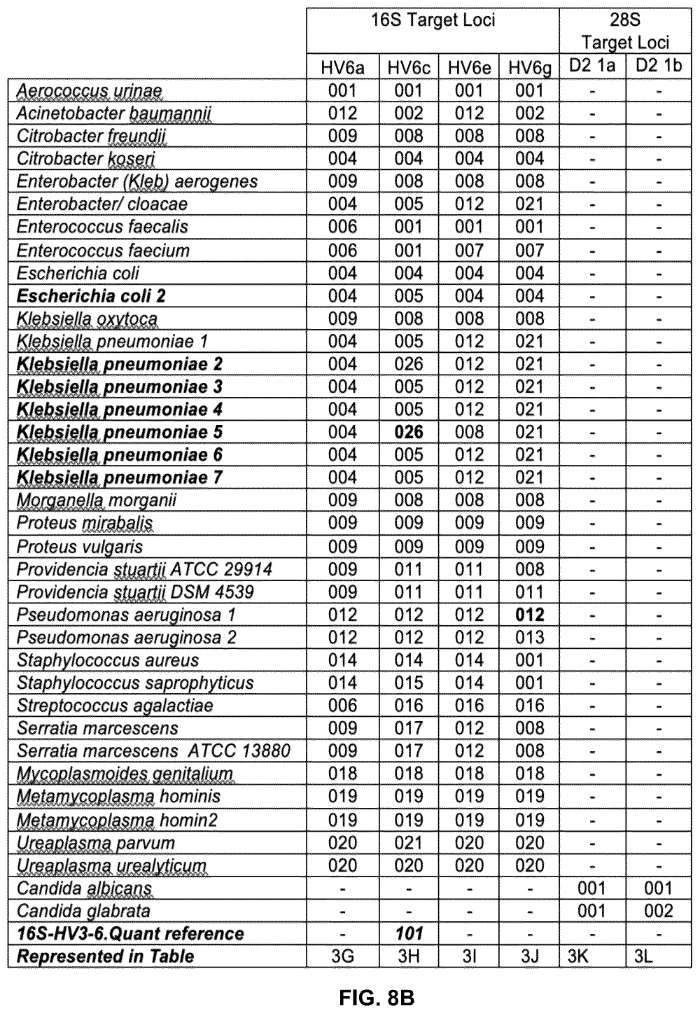

Multiplex PCR primer design (Example 1) and microarray hybridization probe design (Example 2) were used to design a PCR+Microarray assay for detection and discrimination of the set of pathogens known to be causative for urinary tract infection (FIG. 2). In the present Example 3, genomic DNA (gDNA) was extracted from well characterized bacterial cell cultures (Zeptometrix, ATCC) and used for multiplex PCR amplification at a template input of 10⁺³ cp/RXN. Subsequent to PCR amplification the resulting asymmetric multiplex PCR product, comprising bacterial 16S Hypervariable region, i.e., HV1, HV3 and HV6, amplicons were subjected to standard 2 hr microarray hybridization at room temperature (FIGS. 2-3) to a microarray containing the probes of Tables 3A-3L without the updated probe sequences (see Example 7, FIGS. 8A-8B) printed in triplicate as a 21×21 microarray (5 mm×5 mm) with one microarray fabricated per well in a standard 96-well format (FIG. 3).

Hybridization was then followed by 15 min of washing at room temp, then imaging of the entire 96-well plate on a Sensospot Imager, using automated image analysis (Augury™). The resulting data comprise the CY-3 fluorescent signal obtained at each probe of the array measured in relative fluorescence units (RFU).

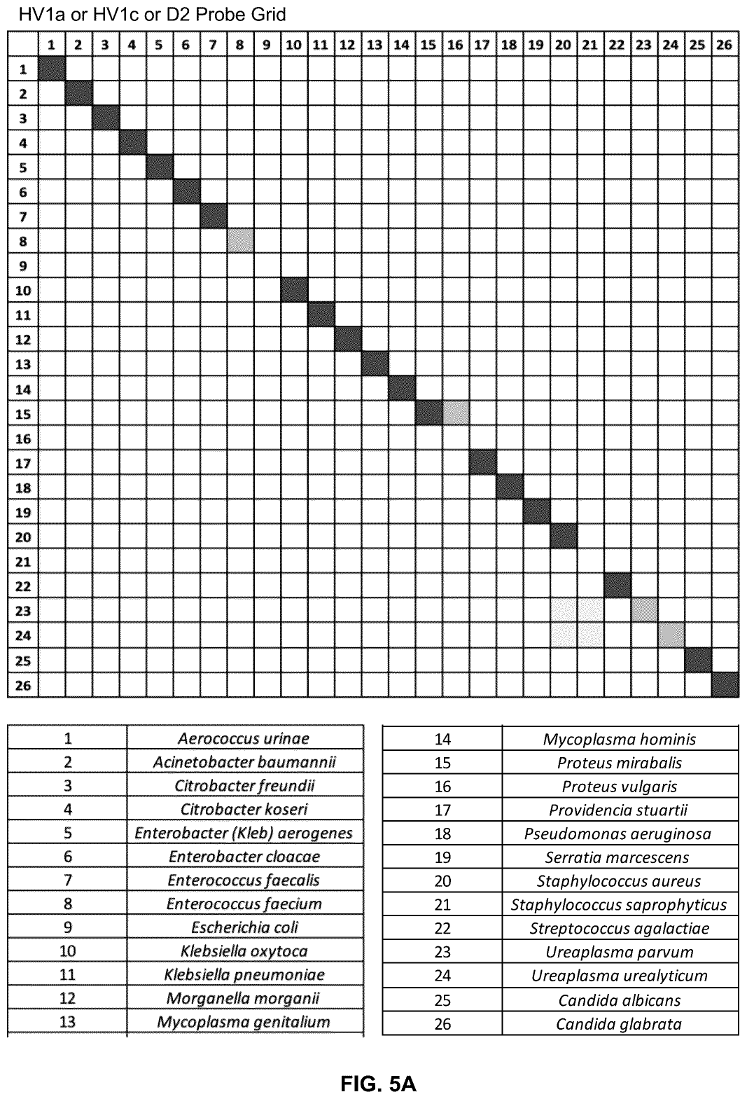

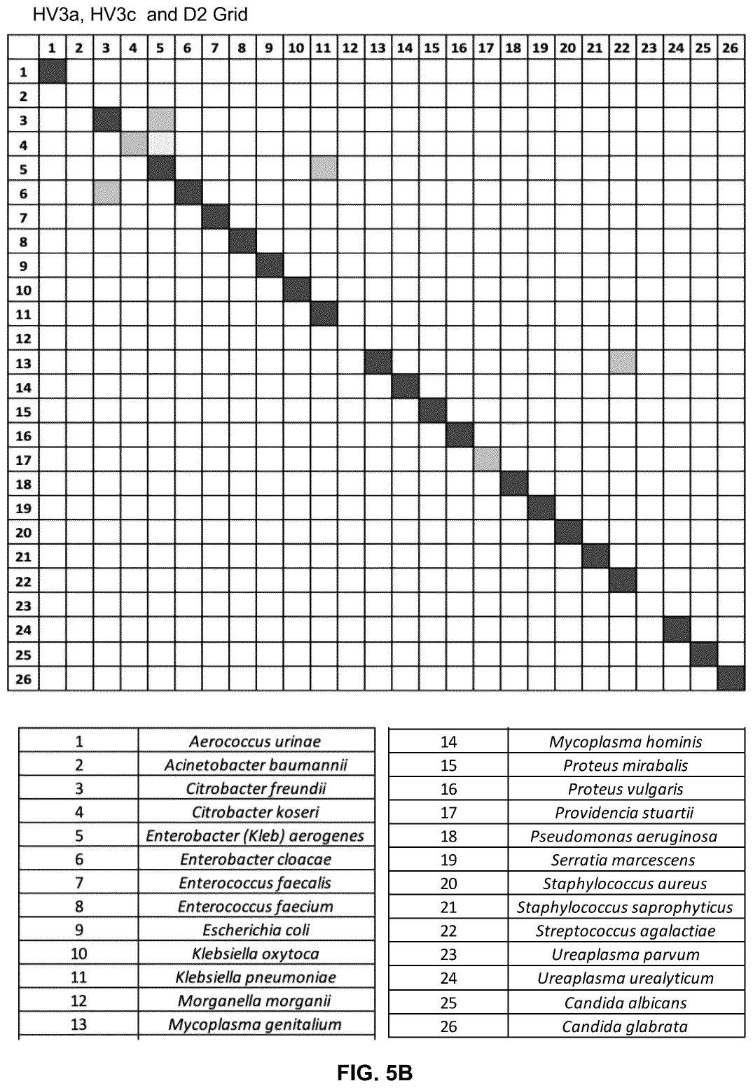

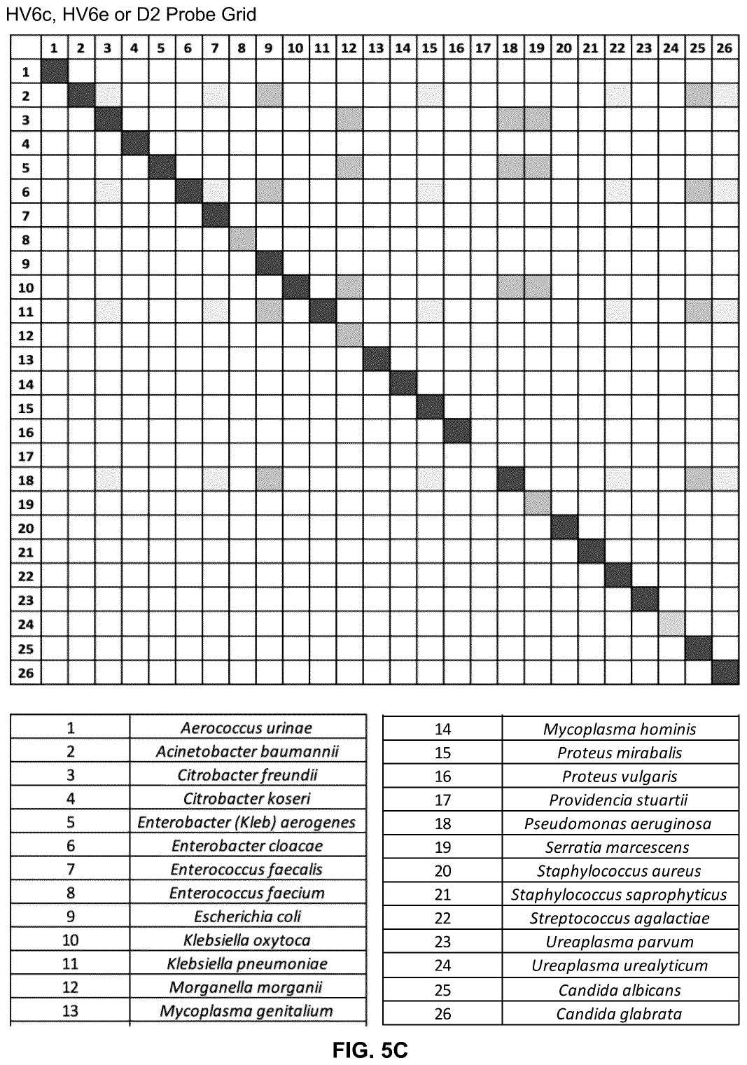

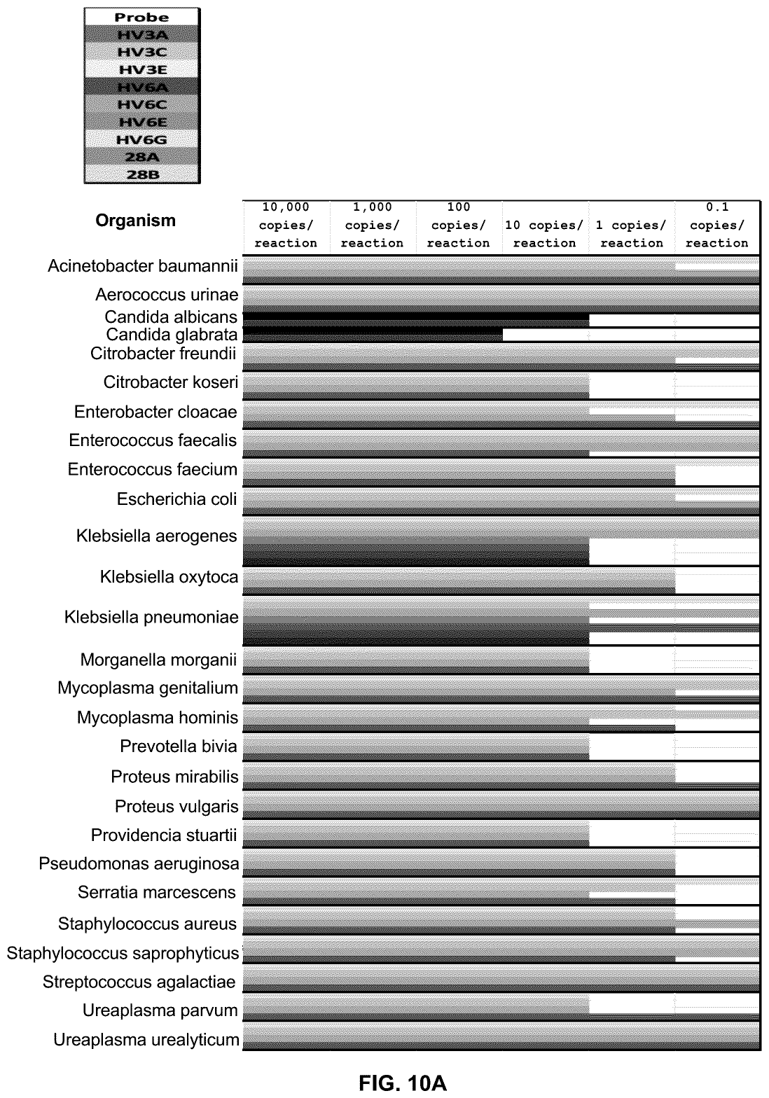

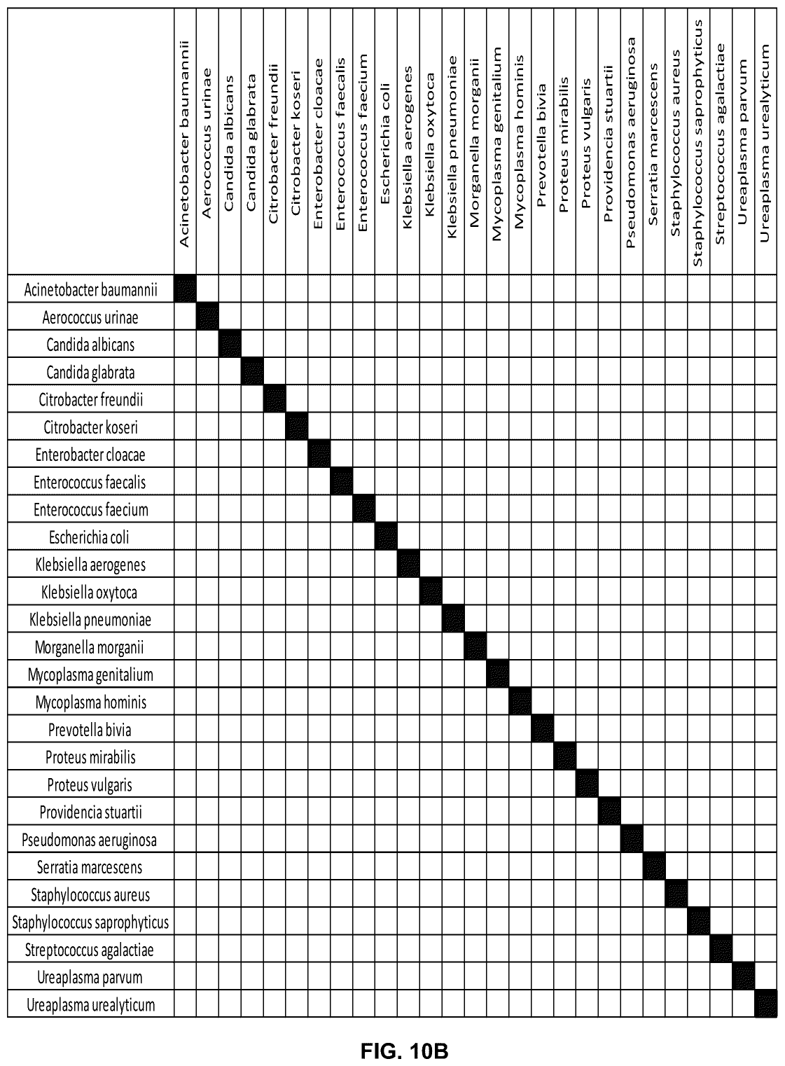

In FIGS. 5A-5C, the outcome of such hybridization analysis is displayed as a matrix, where the Y axis of the 24×24 matrix identifies each of the 24 bacterial pathogens and 2 fungal pathogens of the assay, as previously described in FIG. 2. The X axis comprises the numbering system for the corresponding “Highly discriminating” probe developed for each species at the 16S-HV1 (Tables 3A-3B) or 16S-HV3 (Tables 3C-3E) or 16S-HV6 (Tables 3G-3J) or the 28S-D2 (Table 3K) except for the updated probe sequences (see Example 6, FIG. 10B).

Per a standard naming used throughout, probes designed for species numbered (n) are given the same number (n). Thus in FIG. 2 (Tables 3A-3L), species specific “highly discriminating” probe hybridization signals fall exclusively on the diagonal of each matrix.

The experimentally-derived hybridization data (in RFU) are presented in FIG. 5A (HV1), FIG. 5B (HV3) and FIG. 5C (HV6 and 28S-D2) with a grey scale code. RFU values at 1×-10× background are assigned a gray color. Those greater than 10× background are assigned a black color. Specific data occur on the diagonal (black). Non specific (cross-hybridization) data occur off the diagonal are the light gray color). In those instances where a probe has not yet been designed, the corresponding table elements on the diagonal remain blank, appearing as a gap in the diagonal. Elsewhere, all off diagonal elements where measured signals are <1× background are presented in white, i.e., no measured hybridization.

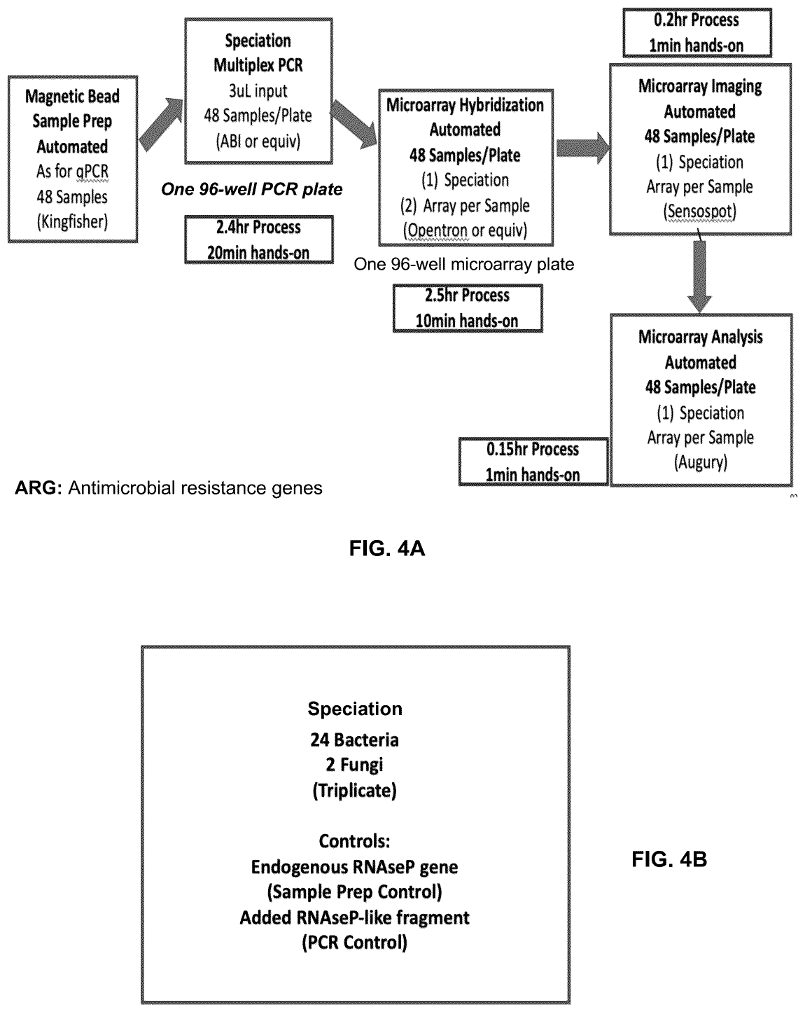

Inspection of the data in FIGS. 5A-5C reveals that hybridization is highly specific, i.e., there are few off diagonal “cross hybridization” events. The data also demonstrate how hybridization among the three hypervariable sites HV1 (FIG. 5A) and HV3 (FIG. 5B) and HV6 (FIG. 5C) is redundant and is used to reinforce the experimental accuracy of the overall data. The miniaturization of the data in FIGS. 5A-5C should be noted. The entirety of the data displayed in FIGS. 5A-5C is obtained in triplicate for each bacterial or fungal sample from a single 5 mm×5 mm microarray placed, one each in each well of a standard 96-well SBS plate. Specifically, each microarray contains 147 highly discriminating probes in triplicate (21×21, 5 mm×5 mm array, FIG. 3) 96-samples may be processed in a single plate on 96 such microarrays in parallel, in about 5 hrs (FIGS. 4A-4B).

FIG. 5A demonstrates that species specific Highly discriminating probes targeting sequence determinants within HV1 may be used to detect and resolve individual species from the representative urinary tract infection syndromic target set described in FIG. 2. Species discrimination is high (few off diagonal elements). FIG. 5B demonstrates that species specific highly discriminating probes targeting sequence determinants within HV3 may be used to detect and resolve individual species from the representative urinary tract infection syndromic target set described in FIG. 2. Species discrimination is high (few off diagonal elements). FIG. 5C demonstrates that species specific Highly discriminating probes targeting sequence determinants within 16S HV6 and 28S D2 may be used to detect and resolve individual species from the representative urinary tract infection syndromic target set described in FIG. 2.

### Example 4

Analytical Limit of Detection Among Microbial Species that are Causative for Uti

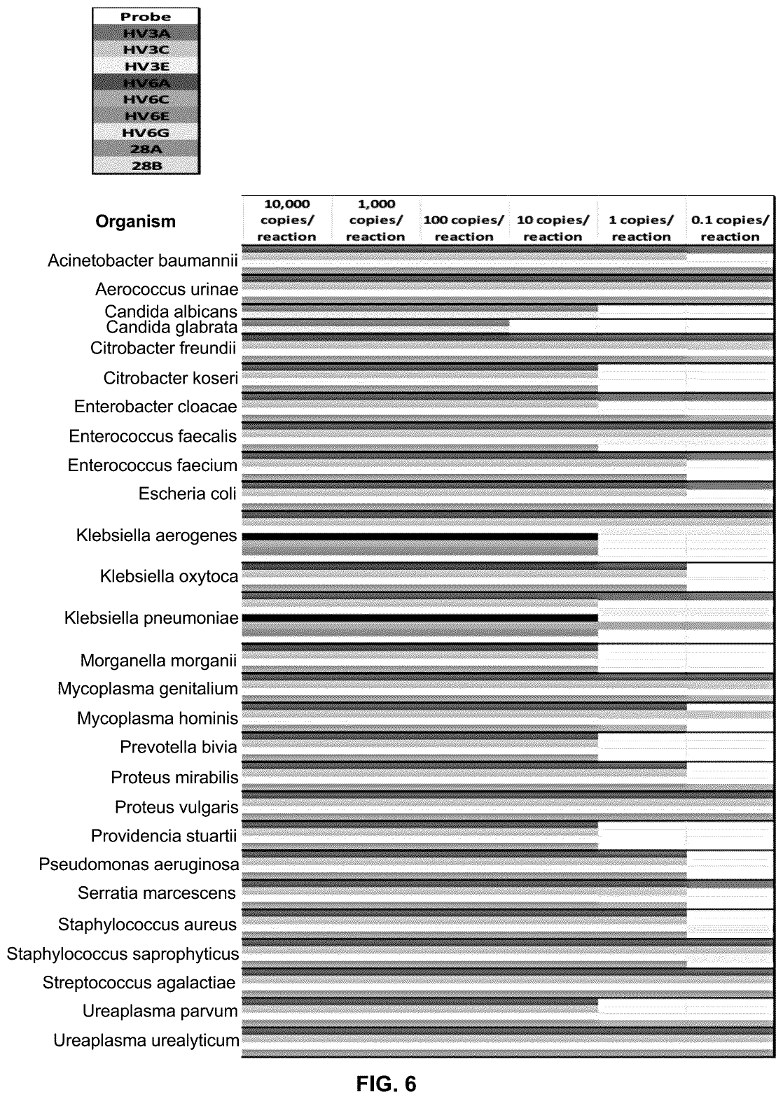

The microarray workflow of FIGS. 4A-4B has been performed on highly characterized purified gDNA over a range of dilution from 10,000 genome copies per Reaction to 0.1 genome copies per reaction. The data of FIG. 6 display a grey scale code for the probes (Tables 3A-3L) to identify each species except for the updated probe sequences (see Example 6, FIG. 10A. At each dilution, if the measured microarray hybridization signal for a probe is determined to be above background, that signal is identified as a continuation of a vertical bar in FIG. 6. At such time that the sample has become too dilute (in terms of genome copies per reaction) to produce a hybridization signal above background at a probe, the coloration of the bar is eliminated, thus identifying the lowest sample (in copies/reaction) which can be detected. In that way, the Limit of Detection (LOD) is identified based on concurrent hybridization to multiple probes, per the present invention. For example, the LOD for most species is determined to be 10 copies/reaction.

### Example 5

### Refinement of Probe and Primer Design to Accommodate Updated Bacterial Sequence Information

As more is learned about sequence heterogeneity within species or as more is learned about Species of Concern that may cause disease, it may be necessary to add or modify the PCR primers and/or the hybridization probes of the present invention, especially those probes which can interrogate such sequence variation within the hypervariable regions of the ribosomal 16S or 28S regions provided as examples in the present invention (see FIGS. 7A-7B for an update of the general structure of FIG. 1).

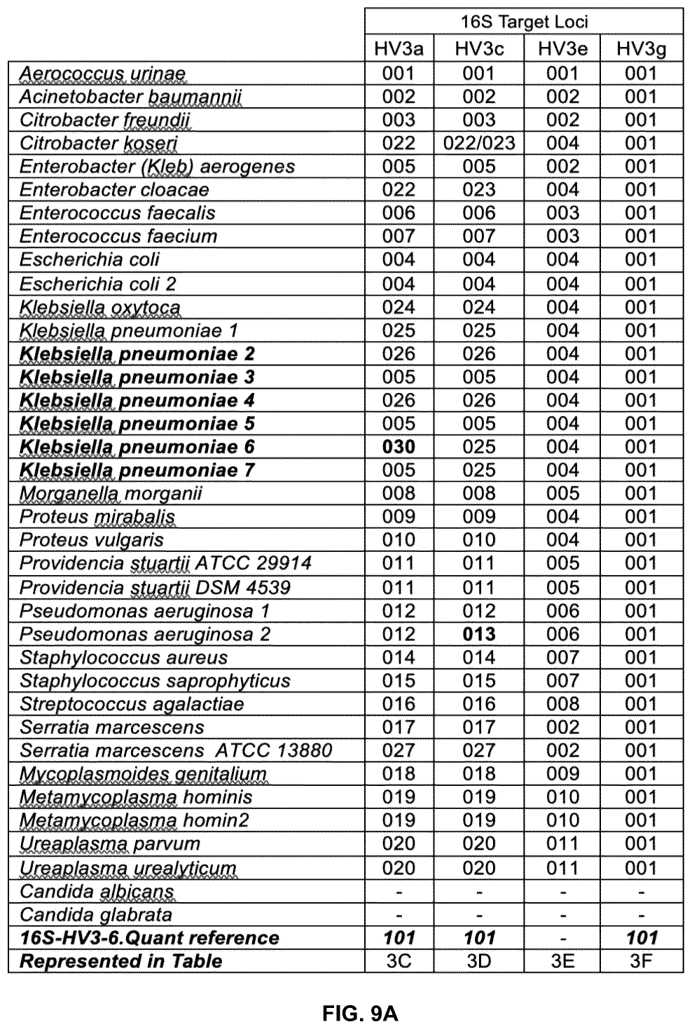

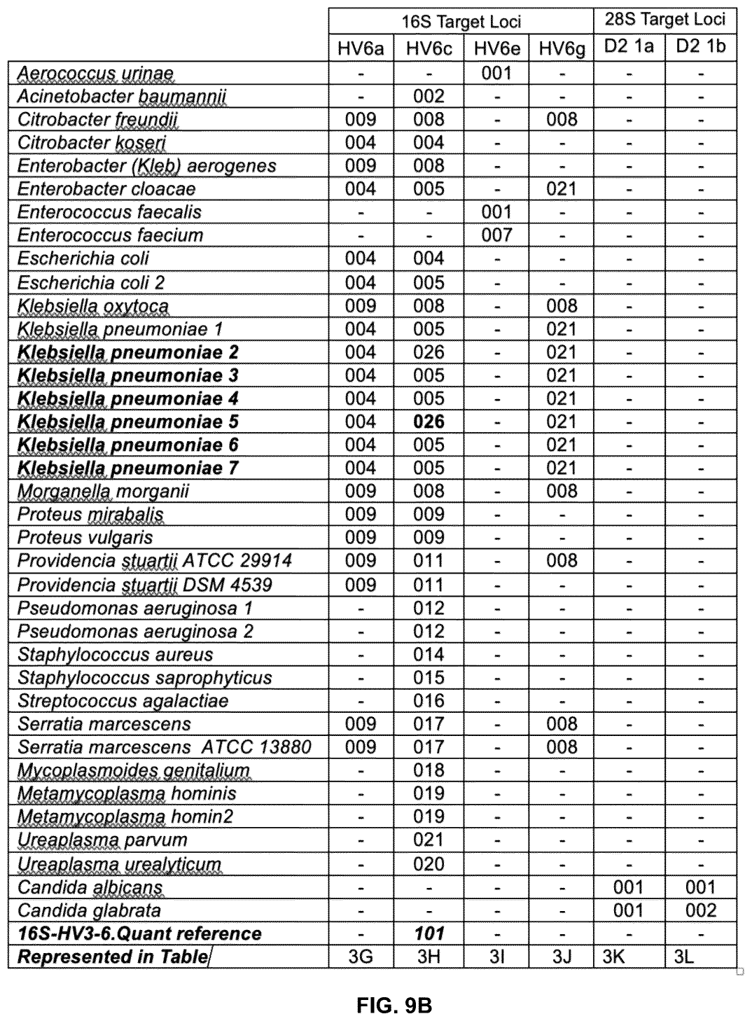

In FIGS. 9A-9B, an update of the hybridization pattern of the present invention shown in FIG. 2 is presented, which changes marked in bold/italic to show how such new knowledge can be accommodated into the present invention. In the present case (bold-italic) is it seen that the specific exemplary changes account for newly identified sequence heterogeneity in E. coli and Klebsiella. Tables 3A-3M display the several specific additions and changes in probe sequence (bold/italic) which accommodate such species heterogeneity or the addition of new Species of Concern. Importantly, such new probe content is introduced into the present invention with only minor additions to the microarray probe layout and little or no change in PCR primer design or number (FIGS. 9A-9B, Tables 3A-3M). Below, it should be noted that with such changes, it is found that the detection and discrimination of Species of Concern in the present invention, for the specific case of UTI infection, may be deployed by PCR amplification and subsequent hybridization analysis of the HV3 and HV6 regions of the 16S rDNA and the analysis of rRNA by RT-PCR (Example 9).

### Example 6

Analytical LOD and Specificity Obtained with Updated Probe and Primer Design Using the Information Content of HV3 and HV6 Hypervariable Region for Bacteria and 28s Region for Fungi

Analytical Specificity was obtained with the update Primer and Probe set (HV3 and HV6) following the same protocol described in Example 3 and is presented in a similar format (FIG. 10A). Therefore, what is shown is the detection of a species based on concurrent analysis of multiple probe sites within HV3 and HV6, such concurrent analysis being performed automatically by software (Augury™) which performs the updated analysis of concurrent probe hybridization (array 1319) as described in FIGS. 9A-9B. As seen in FIG. 10A, at 100 genome copies/RXN, no evidence is seen (i.e., no off diagonal elements shown positive) for any of the Species of Concern of the present UTI deployment of the technology, i.e., no off diagonal elements shown positive. FIG. 10B is a display of experimental aLOD data obtained as described in Example 4, also based on the updated probe content described in Tables 3A-3M (array 1319) and also based on concurrent analysis of HV3 and HV6 amplicons and 28S D2 amplicons. There, it can be seen that the aLOD for all bacterial species falls in the range from 0.1 to 10 copies/RXN. The single outlier is for C. glabrata 100cp/RXN, which is a fungal species.

### Example 7

Clinical Concordance Obtained Vs qPCR on 377 Urine Samples Previously Analyzed by qPCR

377 clinical urine isolates have been obtained as anonymous samples from a CAP-CLIA lab. These samples has been previously subjected to DNA isolation via a combination of MagMax magnetic beads executed on a Kingfisher robot, per the workflow suggested by Thermo Fisher. The resulting purified DNA has been previously analyzed by multiplex qPCR (Thermo Fisher Open Array) for which species detection and discrimination had been obtained for the full set of Species of Concern described in FIGS. 9A-9B and Tables 3A-3M. The workflow of FIGS. 4A-4B was used to obtain species detection and discrimination by the combination of multiplex endpoint PCR amplification (HV3 and HV6) followed by hybridization analysis via the probes and probe array design of FIGS. 9A-9B and Tables 3A-3M. Concordant probe hybridization analysis as described in FIGS. 8A-8B, were performed automatically by Augury Software (FIGS. 4A-4B). Species determination made in that way, per the present invention, was been compared directly to species determination made by the Open Array multiplex qPCR referent. Those data are presented in Tables 4A-4B for all Species of Concern. The 377 sample were analyzed in two set (155 and 192 samples) where it can be seen that the overall concordance between species (present invention vs Open Array) was 97% and 94.5%, respectively.

TABLE 4A
UTI Concordance By Species (155 Samples in Triplicate)

| Organism | Concordance (%) |
|---|---|
| Acinetobacter baumannii | 100% |
| Aerococcus urinae | 85% |
| Candida albicans | 98% |
| Candida glabrata | 95% |
| Citrobacter freundii | 97% |
| Citrobacter koseri | 95% |
| Enterobacter cloacae | 97% |
| Enterococcus faecalis | 97% |
| Enterococcus faecium | 99% |
| Escherichia coli | 99% |
| Klebsiella oxytoca | 100% |
| Klebsiella pneumoniae | 95% |
| Morganella morganii | 94% |
| Proteus mirabilis | 100% |
| Proteus vulgaris | 92% |
| Providencia stuartii | 98% |
| Pseudomonas aeruginosa | 100% |
| Serratia marcescens | 100% |
| Staphylococcus aureus | 98% |
| Staphylococcus saprophyticus | 99% |
| Streptococcus agalactiae | 99% |
| UTI Average Concordance | 97% ± 5% |
| for Clinical Samples | |

TABLE 4B
UTI Concordance By Species (192 Samples in Triplicate)

| Organism | Concordance (%) |
|---|---|
| Acinetobacter baumannii | 96% |
| Aerococcus urinae | 83% |
| Candida albicans | 97% |
| Candida glabrata | 95% |
| Citrobacter freundii | 94% |
| Citrobacter koseri | 93% |
| Enterobacter cloacae | 89% |
| Enterococcus faecalis | 92% |
| Enterococcus faecium | 99% |
| Escherichia coli | 96% |
| Klebsiella oxytoca | 100% |
| Klebsiella pneumoniae | 89% |
| Morganella morganii | 89% |
| Proteus mirabilis | 99% |
| Proteus vulgaris | 92% |
| Providencia stuartii | 98% |
| Pseudomonas aeruginosa | 95% |
| Serratia marcescens | 100% |
| Staphylococcus aureus | 98% |
| Staphylococcus saprophyticus | 99% |
| Streptococcus agalactiae | 96% |
| UTI Average Concordance | 95% +/− 5% |
| for Clinical Samples | |

### Example 8

Introduction of Quantitation into Multiplex Analysis Via the Use of Internal Quantitative Reference Standards for Hypervariable Regions

Quantitation of bacterial pathogens is performed internally within each array of the present invention by hybridization via simultaneous analysis of a synthetic internal ribosomal gene standard which contains the consensus sequence of its cognate HV regions (bacterial or fungal). Thus, if added to a PCR or RT-PCR master mix, the standard is amplified in parallel with the corresponding pathogen nucleic acid present in the same reaction (FIGS. 11A-11C). The sequence of the standard is designed to be 99+% identical to each pathogen. For bacterial quantitation, the standard is a 790 bp artificial “g-Block” gene fragment spanning 16S rDNA region containing both HV3 and HV6 (Table 3N) and thus may be used as a standard for both amplicons. The standard is designed to contain about 1% of base changes relative to the natural sequence. Those changes are targeted to the regions used for probe design that display the largest range of sequence diversity, namely HVa, HV3C and HV6c). The resulting standard-specific probes are given the suffix (101) and are listed in Tables 3C, 3D, 3F, and 3H, in the final row of each table (bold-italic). Because of the targeted sequence difference at a probe site, the presence of the standard can be resolved by hybridization from that of the closely related natural pathogen present in the same amplification reaction.

Upon completion of PCR or RT-PCR amplification the two amplicon variants “Pathogen” vs “Standard” can be resolved by quantitation of hybridization signals obtained for the two types of probe, pathogen and standard as described in Table 5 and Table 6 (FIG. 11A).

TABLE 5
UNG/RT-PCR reaction conditions

| | | | | Cycle |
|---|---|---|---|---|
| UNG/RT-PCR Reaction Steps | | Temperature | Time | Number |
| UNG Treatment | UNG Treatment | 25° C. | 5 min | 1x |
| First Strand cDNA | Reverse | 55° C. | 20 min | 1x |
| Synthesis | Transcription | | | |
| | Initial | 94° C. | 2 min | 1x |
| | Denaturation | | | |
| PCR Amplification | Denaturation | 94° C. | 30 sec | 40x |
| | Annealing | 55° C. | 30 sec | |
| | Extension | 68° C. | 30 sec | |
| | Final Extension | 68° C. | 7 min | 1x |

| | | |
|---|---|---|
| Hold | 4° C. | ∞ |

TABLE 6
PCR Speciation Primer

| | | Conc in |
|---|---|---|
| Primer | Sequence 5′-3′ | reaction (nM) |
| 28S FP 1.1 | TTGTACGTGAAATTGTTGAAAG | 150 |
| | (SEQ ID NO: 8) | |
| 28S RP C3 1.1 | /5Cy3/TTAAACCGCAGTCCTCRGTC | 600 |
| | (SEQ ID NO: 9) | |
| 16S HV3 FP 1.1 | TTTCACATTGGRACTGAGACACGG | 200 |
| | (SEQ ID NO: 4) | |
| 16S HV3 RP C3 1.1 | /5Cy3/TTTTGTATTACCGCGGCTGCTGGCA | 800 |
| | (SEQ ID NO: 5) | |
| 16S HV6 FP 1.1 | TTCGCACAAGCGGTGGAGCATGT | 100 |
| | (SEQ ID NO: 6) | |
| 16S HV6 RP C3 2.1 | /5Cy3/TTTACGAGCTGACGACAGCCATGCA | 400 |
| | (SEQ ID NO: 7) | |
| RNaseP FP 1.1 | TTTGTTTGCAGATTTGGACCTGCGAGCG | 5 |
| | (SEQ ID NO: 10) | |
| RNaseP RP C3 1.1 | 5Cy3/TTTAAGGTGAGCGGCTGTCTCCACA | 20 |
| | AGT (SEQ ID NO: 11) | |
| 16S-HV3-6.Quant ref | TABLE 3N (SEQ ID NO: 173) | 5000 |
| | | Copies |
| | | per Reaction |

Details of the process are as follows. The standard undergoes PCR amplification with the same efficiency as matched bacterial or fungal pathogens in the same sample via identical PCR priming sites in the conserved 16S rDNA or 28s rDNA flanking regions. In the present example, the standard's PCR product hybridizes to engineered HV3a, HV3c and HV6c probes complementary to its slightly altered 16sDNA sequence (Tables 3A, 3B, 3H), At least 3 independent measurements of these Pathogen/Standard hybridization rations are measured for each Species of Concern resident in such a test. Thus, a quantitative measure is obtained, in parallel, for all species detected in the assay.

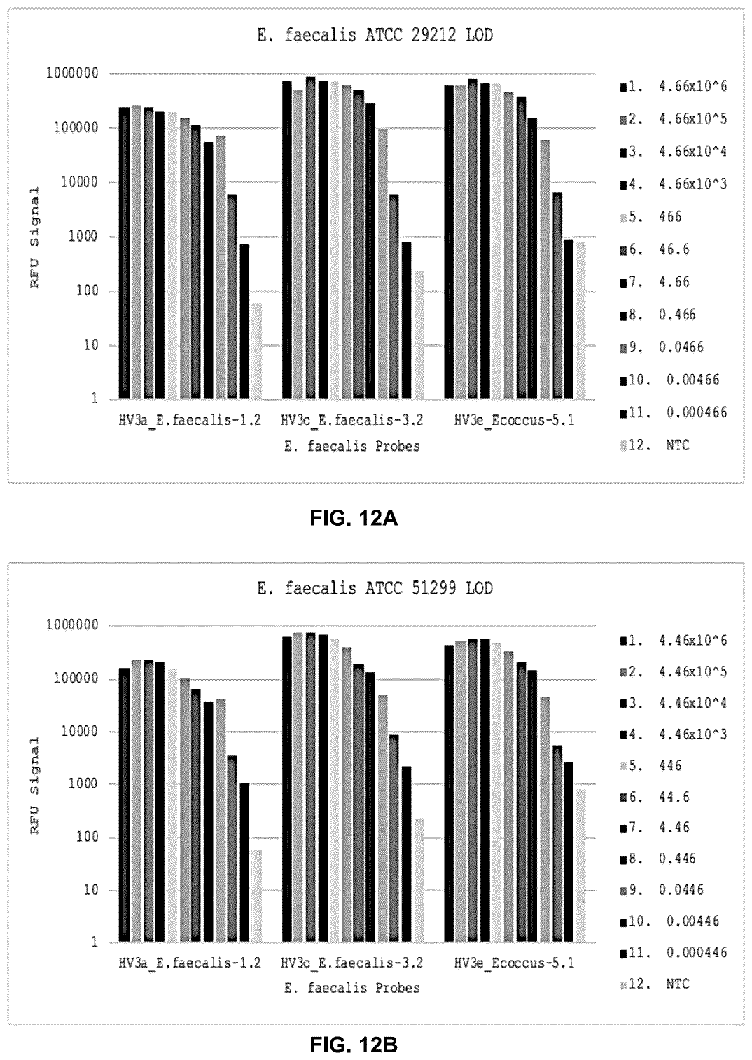

The seminal data quantity is the Hyb ratio [Pathogen/Standard] which is related to the number of DNA molecules (and in turn cell equivalents) for each pathogen in a sample relative to the known number of standard molecules introduced per reaction, which is 5000 in Table 6 and FIGS. 12A-12B. The Hyb ratio is calculated autonomously by Augury software (FIGS. 4A-4B). The relationship between the Hyb Ratio and the number of Pathogen cells in a reaction are determined via validation studies such as those as shown in FIG. 11B and FIG. 11C. There, it is shown that the measured Hyb Ratio measured at multiple sites for each species (HV3a, HV3c, HV6a) is found to be linear (on a log scale) with added pathogen cell number about a 4 log range [0.01 to 100] and thus is readily fit by a log-linear regression to enable quantitation constants. Such fits of experimental gDNA titrations are shown in FIG. 11B and FIG. 11C for two different Species of Concern of the representative assay defined in FIGS. 8A-8B and FIGS. 9A-9B. Quantitation via this “Hyb Ratio” methods can be directly compared to Quantitation by qPCR (Cq) or other methods of nucleic acid quantitation and covers a similar practical dynamic range: 10²-10⁶ cell equivalents of Pathogen DNA/RXN.

### Example 9

Ribosomal RNA (rRNA) Analysis by RT-PCR Amplification Followed by Array Hybridization Analysis

Example 9 provides a reduction to practice showing that rRNA, followed by Rt-PCR amplification, can be used as the Pathogen nucleic acid target in the present invention. For this first RT-PCR deployment, a pair of highly standardized E. faecalis bacterial cell lines ATCC 29212: Vancomycin Susceptible were chosen because it lacks van A/B antibiotic resistance genes) and ATCC 51299: Vancomycin Resistant because it contains a vanB gene). These two lines have been validated by CLSI (the Clinical Lab Safety Institute) and are used widely as standards for antibiotic susceptibility testing in CLIA labs. For the present example, they have both been expanded by fluid culture to mid log phase (Brain Heart Infusion Broth) harvested and enumerated by analytical plate culture using Brain Heart Infusion Agar Plates. The OD600 was also measured post overnight culture to estimate a cell/mL value for each. Total RNA was then extracted using a standard Zymo Bacterial RNA kit, including DNA removal with DNAse1 per manufacturer instructions: Zymo Quick-DNA/RNA MagBead kit catalog number R2130. For the two E. faecalis cell lines, the resulting total RNA preparation was then quantified by UV/VIS (nanodrop) with the resulting concentration converted to cell equivalents per unit volume assuming @100% recovery of RNA from the original enumerated cell preparation. Based on that quantitation, Serial dilutions were prepared to yield a total RNA input per RT-PCR RXN (5 uL/50 uL RXN) equivalent to the following 10-step dilution series [4.66×10⁺⁶, 4.66×10⁺⁵, 4.66×10⁺⁴, 4.66×10⁺³, 466, 46, 4.66, 0.466, 0.046 and 0] cell equivalents per RT-PCR reaction. An endpoint RT-PCR reaction was performed on each with cycling conditions (Table 5) very similar to the analogous endpoint PCR reaction used for rDNA (Table 1) with RT-PCR primers also identical to those used for PCR (Table 6). Subsequent to endpoint PCR, array hybridization was performed via the same microarray workflow used for PCR (FIGS. 4A-4B) on a microarray with probes identical to that used for analysis of rDNA by PCR (Tables 3A-3L).

The raw hybridization results of those titrations are displayed in FIGS. 12A-12B as a range finding aLOD experiment i.e., hybridization signal in Relative Fluorescent Units (RFU) plotted on a log scale vs RT-PCR sample input in cell equivalents/RXN. Data are presented for all three HV3 specific hybridization probes necessary to uniquely identify E. faecalis (see FIGS. 8A-8B, FIGS. 9A-9B). As seen, signal strength for all three probe signals remains above the 0 cell/RXN threshold signal (NTC) at or above 0.04 cell equivalents/RXN, Thus, visual inspection of the data suggest that the apparent aLOD for both E. faecalis cell lines is at @0.05 cell equivalents/reaction.

In order to confirm that approximate aLOD and to interrogate the specificity of the analysis, all data on the 21×21 1319 array were submitted to concurrent hybridization analysis via Augury software (Table 7 shows representative Augury data output), over the entire titration range, for both E. faecalis lines see (Table 8).

TABLE 7
RFU results of RT-PCR

| | | |
|---|---|---|
| Well Number: | 1 | |
| Sample Name: | Sample #1 E. faecalis ATCC | |
| | 29212 | |
| ** Controls: ** | 4.66x 10 + 6 Cells/RXN | |
| | Negative Control | Not Detected |
| | RNaseP Control | Not Detected |
| ** Pathogens/Genes | Enterococcus faecalis | |
| Detected: | Candida glabrata | |
| Well Number: | 5 | |
| Sample Name: | Sample #5 E. faecalis ATCC | |
| | 29212 | |
| ** Controls: ** | 4.66x 10 + 2 Cells/RXN | |
| | Negative Control | Not Detected |
| | RNaseP Control | Not Detected |
| ** Pathogens/Genes | Enterococcus faecalis | |
| Detected: | | |
| Well Number: | 11 | |
| Sample Name: | Sample #11 E. faecalis ATCC | |
| | 29212 | |
| ** Controls: ** | 0.000466 Cells/RXN | |
| | Negative Control | Not Detected |
| | RNaseP Control | Not Detected |
| ** Pathogens/Genes | No Listed Pathogens Detected | |
| Detected: ** | | |
| Well Number: | 12 | |
| Sample Name: | Sample #12 NTC | |
| ** Controls: ** | Negative Control | Not Detected |
| | RNaseP Control | Not Detected |
| ** Pathogens/Genes | No Listed Pathogens Detected | |
| Detected: | | |

Detection and Discrimination of these two well characterized E. faecalis (CLSI) standards was obtained over the entire 7-log range of input rRNA density (Table 8). Data obtained from this initial autonomous (Augury™) range finding analysis (Table 8) suggest an aLOD of <0.05 cell equivalent of rRNA for both lines, in good agreement with the visual inspection of the raw RFU data (Tables 7-8; FIGS. 12A-12B).

TABLE 8

| | E faecalis | | | E faecalis | |
|---|---|---|---|---|---|
| | ATCC 29212 | | | ATCC 51299 | |
| | vancomycin | | | Vancomycin | |
| | susceptible | Augury | | Resistant | Augury |
| Sample | (genome lacks | Analysis | Sample | (genome has | Analysis |
| ID | van genes) | (Autonomous) | ID | vanB gene) | (Autonomous) |
| 1 | 4.66x 10 + 6 | Enterococcus | 1 | 4.46x 10 + 6 | Enterococcus |
| | Cells/RXN | faecalis & | | Cells/RXN | faecalis & |
| | | Candida | | | Candida |
| | | glabrata | | | glabrata |
| | | Detected | | | Detected |
| 2 | 4.66x 10 + 5 | Enterococcus | 2 | 4.46x 10 + 5 | Enterococcus |
| | Cells/RXN | faecalis & | | Cells/RXN | faecalis & |
| | | Candida | | | Candida |
| | | glabrata | | | glabrata |
| | | Detected | | | Detected |
| 3 | 4.66x 10 + 4 | Enterococcus | 3 | 4.46x 10 + 4 | Enterococcus |
| | Cells/RXN | faecalis | | Cells/RXN | faecalis |
| | | Detected″ | | | Detected |
| 4 | 4.66× 10 + 3 | Enterococcus | 4 | 4.46x 10 + 3 | Enterococcus |
| | Cells/RXN | faecalis | | Cells/RXN | faecalis |
| | | Detected″ | | | Detected |
| 5 | 4.66x 10 + 2 | Enterococcus | 5 | 4.46x 10 + 2 | Enterococcus |
| | Cells/RXN | faecalis | | Cells/RXN | faecalis |
| | | Detected | | | Detected |
| 6 | 46.6 Cells/RXN | Enterococcus | 6 | 44.6 Cells/RXN | Enterococcus |
| | | faecalis | | | faecalis |
| | | Detected | | | Detected |
| 7 | 4.66 Cells/RXN | Enterococcus | 7 | 4.46 Cells/RXN | Enterococcus |
| | | faecalis | | | faecalis |
| | | Detected | | | Detected |
| 8 | 0.466 | Enterococcus | 8 | 0.446 | Enterococcus |
| | Cells/RXN | faecalis | | Cells/RXN | faecalis |
| | | Detected | | | Detected |
| 9 | 0.0466 | Enterococcus | 9 | 0.0446 | Enterococcus |
| | Cells/RXN | faecalis | | Cells/RXN | faecalis |
| | | Detected | | | Detected |
| 10 | 0.00466 | No listed | 10 | 0.00446 | No listed |
| | Cells/RXN | pathogens | | Cells/RXN | pathogens |
| | | Detected | | | Detected |
| 11 | 0.000466 | No listed | 11 | 0.000446 | No listed |
| | Cells/RXN | pathogens | | Cells/RXN | pathogens |
| | | Detected | | | Detected |
| 12 | No added | No listed | 12 | No added | No listed |
| | Cells.RXN | pathogens | | Cells.RXN | pathogens |
| | (NTC) | Detected | | (NTC) | Detected |

Autonomous Augury™ software analysis shows that the only bacterial species consistent with the observed pattern of hybridization was that of E. faecalis, even up to a 10⁺⁶ excess of input RNA above that of the apparent aLOD, suggesting excellent Discrimination. Interestingly at the two highest inputs (4.66×10+6, 4.66×10+5 cells/RXN) C. glabrata (a fungi) was also detected, selectively as distinct from C. albicans, which is also among the Species of Concern. It is likely that the presence of C. glabrata detected at very high sample input (>10⁺⁵ cells/RXN) may be real and due to small, but measurable trace contamination in the RNA preparation.

### Example 10

### Multiplex Isothermal NASBA Amplification and UTI Array Detection of Eight ESKAPE++Pathogens

rRNA was extracted from cells (Table 9) purchased from Zeptometrix using the PDx OCTA automated system per manufacturers IFU. NASBA amplification was done using NASBA kit NWK-1 from Life Sciences Advanced Technologies Inc. following manufacturer's instruction. NASBA primers were customized for multiplex NASBA amplification to produce short HV3 and HV6 amplicons (Table 10). The principles used for such NASBA primer design have been previously described in a companion patent awarded U.S. Pat. No. 12,054,794 B2 issued Aug. 6, 2024 which is incorporated herein by reference.

TABLE 9
Cell lines used in multiplex NASBA-D3 Array

| | | | Cell Titer |
|---|---|---|---|
| Organism from Zeptometrix Cells | Catalog No. | Lot Number | CFU/mL |
| Enterococcus faecium Z347, vanA | 0801892 | 331582 | 2.85 × 10\hat{ }9 |
| Staphylococcus aureus Z482, tittered | 0804125 | 331471 | 1.05 × 10\hat{ }9 |
| Klebsiella pneumoniae Z138, | 0801507 | 331468 | 6.92 × 10\hat{ }9 |
| OXA-48, CTX-M | | | |
| Acinetobacter baumannii 307-0294 | 0801597 | 330008 | 1.36 × 10\hat{ }9 |
| Pseudomonas aeruginosa Z189, VIM-1 | 0801908 | 330523 | 3.78 × 10\hat{ }8 |
| Escherichia coli Z136, CTX-M-15 | 0801905 | 329319 | 7.17 × 10\hat{ }9 |
| Klebsiella aerogenes Z052 | 0801518 | 330468 | 1.33 × 10\hat{ }10 |
| Enterobacter cloacae Z101 | 0801830 | 330006 | 1.30 × 10\hat{ }10 |

Hybridization and Detection conditions were taken directly from the PCR based UTI array assay (FIGS. 4A-4B). Sequences of the multiplex primers, detector oligos, and detecting probes are shown in Table 10. Total amplification+Hybridization time=3.5 hrs.

TABLE 10
NASBA primers and probes for detection of ESKAPE pathogens
Amplification primers for the isothermal PDx-ESK+ test
(modified from PCR primer sequences on D3 Array-UTI)

| | Forward (5′→3′) | Reverse (5′→3′) |
|---|---|---|
| | Underlined = | Small letters = T7 promoter |
| | Detector oligo | sequence |
| | binding site | Underlined = |
| | 16S rDNA (HV3 | Detector oligo |
| | short-A&B) | binding site |
| 16S rDNA | TTTCTACCGTACTCTAGCT.CC | AATT taatacgactcactataggg |
| (HV3 short-A&B) | ATGCCGCGTGWGTGAAGAAG | ATAA CTCAACAG TGTATTA |
| Amplicon | G SEQ ID NO: 193 | CCGCGGCTGCTGGCA |
| Len = 177 bases | TTTCTACCGTACTCTAGCT.CA | SEQ ID NO: 195 |
| | ACGCCGCGTGAGTGATGAAGG | |
| | SEQ ID NO: 194 | |
| 16S rDNA | TTTCTACCGTACTCTAGCT | AATT taatacgactcactataggg |
| (HV3 short-A&B) | GAGCATGTGGTTTAATYCGA | ATAA CTCAACAG |
| Amplicon | SEQ ID NO: 196 | AGCTGACGACAYCCATGCA |
| Len = 177 bases | | SEQ ID NO: 197 |

Biotinylated detector oligos

| | | | |
|---|---|---|---|
| FP detector | 5′Biotin- | “T7” | 5′Biotin-x.CTGTTGAG |
| | x.TCTACCGT | detector | TTATCCT.x |
| | ACTCTAGCT.x | | SEQ ID NO: 199 |
| | SEQ ID NO: 198 | | |

Microarray hybridization probes for the isothermal PDx-ESK+ test
(identical to those probes presently on D3 Array-UTI)

| | HV3a probe | HV3c probe | HV3e probe | HV6c probe |
|---|---|---|---|---|
| Bacterium | (5′-3′) | (5′-3′) | (5′-3′) | (5′-3′) |
| E. faecium | | (007)-x | | |
| | | TAACTGTTCAT | | |
| | | CCCTTG | | |
| | | x | | |
| | | SEQ ID | | |
| | | NO: 200 | | |
| S. aureus | | (014)-x | | |
| | | TGTG | | |
| | | CACATCTTGAC | | |
| | | x | | |
| | | SEQ ID | | |
| | | NO: 201 | | |
| K. pneumoniae 1 | | | | (005)-x |
| | | | | CCACAGAAC |
| | | | | TTTCC |
| | | | | x |
| | | | | SEQ ID |
| | | | | NO: 202 |
| A. baumannii | (002)-x | | | |
| | GCTACTTTA | | | |
| | G TTAATAC | | | |
| | x | | | |
| | SEQ ID | | | |
| | NO: 203 | | | |
| P. aeruginosa | | | (006)-x | |
| | | | CGTTACCAA | |
| | | | CAGAATA | |
| | | | x | |
| | | | SEQ ID | |
| | | | NO: 204 | |
| Enterobacter | (022)-x | | | |
| spp | AAGGTGTTG | | | |
| (cloacae) | TGGTTA | | | |
| | x | | | |
| | SEQ ID | | | |
| | NO: 205 | | | |
| + K. aerogenes | | | | (008)-x |
| | | | | CCAGAGAAC |
| | | | | TTAGC x |
| | | | | SEQ ID NO: 206 |
| + E. coli | | (004)-x TACC | | |
| | | TTTGCTCATTG | | |
| | | A x SEQ ID NO: 207 | | |

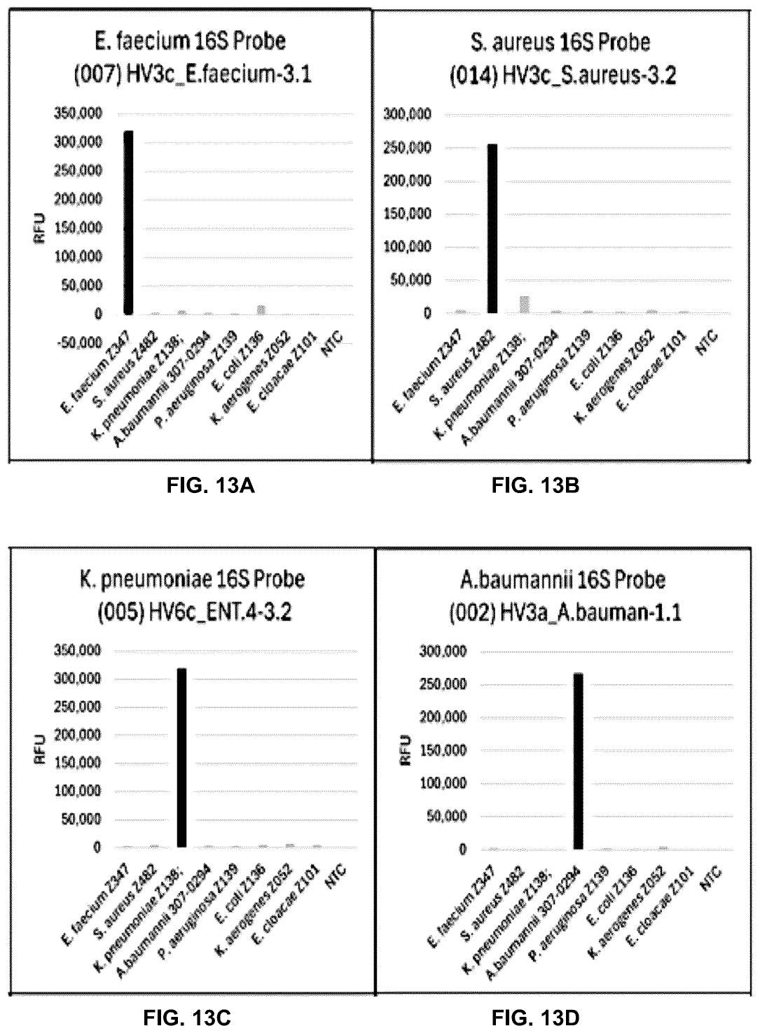

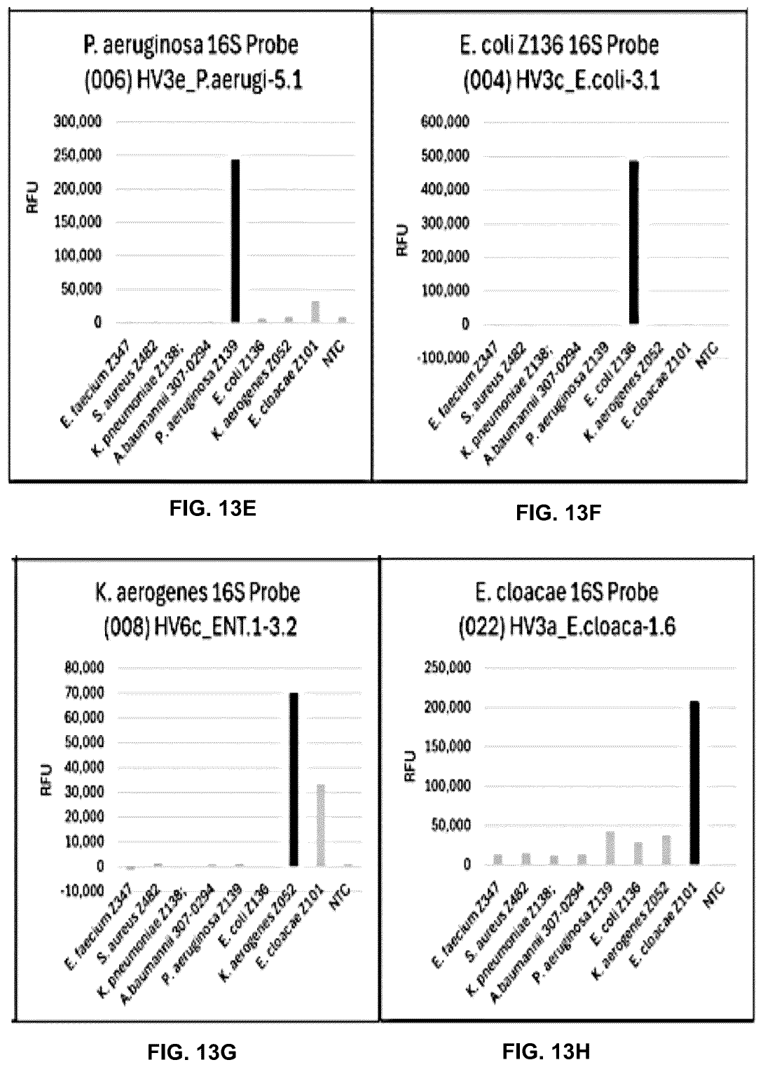

Result: Data shown in FIGS. 13A-13H are the average of 4 wells: i.e. hybridization analysis in triplicate for all eight ESKAPE₊₊ pathogens in each of 4 wells of the D3 Array test. Black bars=Averaged Hybridization Signals (in RFU) detected by the predicted species-specific probes for each spiked ESKAPE₊₊ bacterium; Gray bars=Cross reaction signals detected by species-specific probes in the ESKAPE₊₊ set NOT SPECIFIC for the spiked in bacteria of interest in each sample. As seen, the species specificity obtained by multiplex NASBA amplification of rRNA hypervariable regions HV3 and HV6 can be revealed by either HV3 or HV6 probe hybridization.

bacterial species and/or fungal species in the sample.

What is claimed:

1. A method for detecting and discriminating among multiple bacterial species and fungal species in a sample, comprising:
obtaining a sample from a subject;
harvesting bacteria and fungi from the sample;
isolating nucleic acids from the harvested bacteria and fungi;
amplifying bacterial and/or fungal ribosomal DNA or ribosomal RNA using at least two fluorescently-labeled primer pairs selective for bacterial and/or fungal hypervariable regions to generate at least two fluorescently labeled ribosomal nucleic acid amplicons;
hybridizing the fluorescently-labeled ribosomal nucleic acid amplicons to a plurality of nucleic acid probes, each probe in said plurality having a sequence complementary to a sequence determinant in one of the amplified ribosomal nucleic acid hypervariable regions and each probe attached to a microarray;
washing the microarray at least once; and
imaging the microarray to detect a fluorescent signal from each of the two or more fluorescently-labeled ribosomal gene amplicons hybridized to the complementary probe; and
analyzing concurrently at least two hypervariable regions, thereby detecting the bacterial species and/or fungal species in the sample.

2. The method of claim 1, further comprising:
amplifying a region of interest in at least one additional gene using at least one fluorescently-labeled primer pair selective for the region of interest to generate at least one additional fluorescently labeled amplicon;
hybridizing the at least one additional fluorescently labeled amplicon, said plurality of nucleic acid probes comprising at least one additional probe having a sequence complementary to the region of interest; and
imaging the microarray to detect a fluorescent signal from each of the at least one additional fluorescently-labeled amplicons hybridized to the complementary probe.

3. The method of claim 2, wherein one of the at least one additional genes comprises human RNaseP as an internal standard.

4. The method of claim 3, wherein the fluorescently-labeled primer pair for the human RNaseP gene has a nucleotide sequence of SEQ ID NOS: 10-11.

5. The method of claim 3, wherein the human RNaseP probe has a nucleic acid of SEQ ID NO: 171.

6. The method of claim 2, wherein the at least one additional genes comprise genes that confer antibiotic resistance to the bacterial species.

7. The method of claim 6, wherein the antibiotic resistant bacterial species are Enterococcus faecium, Staphylococcus aureus, Klebsiella pneumoniae, Acinetobacter baumannii, Pseudomonas aeruginosa, Enterobacter cloacae, Escherichia coli and Klebsiella aerogenes.

8. The method of claim 1, further comprising quantifying an abundance of each species in the sample.

9. The method of claim 8, comprising:
adding a known number of copies of a quantitative reference standard to the amplifying step wherein said quantitative reference standard has a nucleotide sequence similar to the nucleotide sequences of the hypervariable regions that are amplified;
adding to the microarray a probe with a sequence complementary to the nucleotide sequence of the quantitative reference standard;
hybridizing the quantitative reference standard to the complementary probe;
measuring a ratio of the hybridization signal in relative fluorescence units from the fluorescently-labeled amplicons hybridized to their complementary probes to a hybridization signal in relative fluorescence units from the quantitative reference standard hybridized to its complementary probe; and
correlating uniquely the ratio to a gene copy number of bacterial species or fungal species in the sample relative to the number of copies of the quantitative reference standard.

10. The method of claim 9, wherein the quantitative reference standard has a probe nucleotide sequence of SEQ ID NO: 173.

11. The method of claim 1, wherein the fluorescently-labeled primer pair comprises a pair of nucleotide sequences that targets a bacterial 16S rDNA hypervariable region or a 28S D2 region, said fluorescently-labeled primer pair selected from the group consisting of SEQ ID NOS: 1 and 2, SEQ ID NOS: 1 and 3, SEQ ID NOS: 4 and 5, SEQ ID NOS: 6 and 7, SEQ ID NOS: 8 and 9 and a combination thereof.

12. The method of claim 1, wherein the plurality of hybridization probes comprises a set of bacterial probes each with a nucleotide sequence corresponding to the sequence determinant in a 16S hypervariable region 1 selected from the group consisting of SEQ ID NOS: 12-31, SEQ ID NOS: 32-50 and a combination thereof.

13. The method of claim 12, wherein the plurality of hybridization probes further comprises a set of bacterial probes each with a nucleotide sequence corresponding to the sequence determinant in a 16S hypervariable region 3 selected from the group consisting of SEQ ID NOS: 51-75, SEQ ID NOS: 76-100, SEQ ID NOS: 101-111, SEQ ID NOS: 112-113 and a combination thereof.

14. The method of claim 13, wherein the plurality of hybridization probes further comprises a set of bacterial probes each with a nucleotide sequence corresponding to the sequence determinant in a 16S hypervariable region 6 selected from the group consisting of SEQ ID NOS: 114-122, SEQ ID NOS: 123-140, SEQ ID NOS: 141-152, SEQ ID NOS: 153-166 and a combination thereof.

15. The method of claim 14, wherein the plurality of hybridization probes further comprises a set of bacterial probes each with a nucleotide sequence corresponding to the sequence determinant in a 28S D2 region selected from the group consisting of SEQ ID NO: 167, SEQ ID NOS: 168-169 and a combination thereof.

16. The method of claim 1, wherein the bacteria and the fungi are associated with detecting and treating a urinary tract infection, a blood infection or a wound infection.

17. The method of claim 16, wherein the bacteria associated with the urinary tract infection are Acinetobacter baumannii, Aerococcus urinae, Citrobacter freundii, Citrobacter koseri, Enterobacter aerogenes, Enterobacter cloacae, Enterococcus faecalis, Enterococcus faecium, Escherichia coli, Klebsiella oxytoca, Klebsiella pneumoniae, Morganella morganii, Mycoplasma hominis, Mycoplasma genitalium, Proteus mirabilis, Proteus vulgaris, Providencia stuartii, Pseudomonas aeruginosa, Serratia marcescens, Staphylococcus aureus, Staphylococcus saprophyticus, Streptococcus agalactiae, Ureaplasma parvum, or Ureaplasma urealyticum and the fungi associated with the urinary tract infection are Candida albicans or Candida glabrata.

18. The method of claim 1, wherein the subject is a human or other mammal or a plant.

19. The method of claim 1, wherein the sample is a blood sample, a urine sample, a wound swab, a urogenital swab or other equivalent sample type, an air sample, a water sample or a surface swab.

20. The method of claim 1, wherein the amplifying step comprises a PCR amplification, an RT-PCR amplification or an isothermal Transcription Mediated NASBA Amplification of rRNA.
